(19) United States
(12) Patent Application Publication
(10) Pub. No.: US 2026/0331211 A1
(43) Pub. Date: Oct. 22, 2026

(54) PAUSE-MAPPER - PARAMETER-FREE MAPPING OF RNA-POLYMERASE PAUSE DENSITY USING THE RECOGNITION-PHYSICS COHERENCE QUANTUM E_coh Equals 0.090 eV

(71) Applicant: Jonathan Washburn, Austin, TX (US)

(72) Inventor: Jonathan Washburn, Austin, TX (US)

(21) Appl. No.: 19/652,348

(22) Filed: Apr. 20, 2026

Related U.S. Application Data
(60) Provisional application No. 63/792,731, filed on Apr. 22, 2025.

(51) Int. Cl. G16B 5/30; G16B 20/30

(52) U.S. Cl. CPC G16B 5/30; G16B 20/30

(57) ABSTRACT

Computer-implemented methods, systems, and non-transitory computer-readable media are disclosed for deterministic mapping of sequence-dependent pause behavior from a nucleic-acid sequence. A sequence and one or more operating conditions, including temperature and a selected machine profile, are received, and a core ladder index is assigned to sequence positions according to residue identity. An effective ladder index is determined for each position, optionally including deterministic corrections based on neighboring residues, local context, modified residues, or annotation inputs. A position-resolved forward rate is computed as a function of the effective ladder index, a coherence-scale barrier quantity, and the operating conditions, and a dwell time is computed from the forward rate. A pause metric, including a normalized pause density in some embodiments, is generated and output. The framework supports multiple processive molecular machines, comparative analysis, streaming processing, atlas generation, and local or network-accessible implementations.

### CROSS-REFERENCE TO RELATED APPLICATIONS

This application claims the benefit of U.S. Provisional Patent Application No. 63/792,731, filed on Apr. 22, 2025, the entirety of which is incorporated herein by reference to the extent permitted by law.

### BACKGROUND OF THE INVENTION

The present invention relates generally to computational genomics, transcription kinetics, deterministic bioinformatics, and sequence-based pause analysis. More particularly, the invention relates to computer-implemented methods, systems, software, and non-transitory computer-readable media for determining position-resolved polymerase pause behavior from a nucleic-acid sequence and one or more assay conditions, including temperature, without requiring sequence-specific training, statistical fitting, or machine-learning model calibration. In preferred embodiments, the invention is applied to RNA polymerase pause mapping. In other embodiments, the invention is applicable to DNA polymerases, reverse transcriptases, ribosome or codon-based translation models, and other processive molecular machines that advance along a biological sequence and exhibit sequence-dependent variations in translocation rate, dwell time, stalling propensity, or pause density.

Transcription and related biological sequence-reading processes are not uniform in rate along a template. RNA polymerases, DNA polymerases, reverse transcriptases, ribosomes, and related processive molecular complexes can slow, pause, stall, or otherwise exhibit position-dependent kinetic variation as they traverse a sequence. Such variation can affect gene expression timing, co-transcriptional or co-translational folding, coupling between molecular processes, regulatory signaling, editing outcomes, error profiles, product yield, therapeutic quality, and other biologically or commercially significant properties.

Existing approaches for identifying or predicting pause behavior typically rely on statistical inference, fitted dwell-time models, hidden Markov models, machine-learning classifiers, neural-network architectures, or empirical rule systems tuned to a specific organism, enzyme, assay platform, or training corpus. Such methods often require repeated parameter estimation, retraining, convergence checking, hyperparameter selection, or benchmark-specific calibration.

As a result, known methods can be non-deterministic in practical operation, difficult to transfer across enzyme classes or assay conditions, computationally expensive at scale, and difficult to interpret in physically meaningful terms at the level of individual sequence positions.

In many existing approaches, outputs are limited to pause calls, likelihood scores, class probabilities, or empirically fitted dwell parameters that do not arise from a single deterministic barrier model applied position by position across a sequence. Methods built on organism-specific or polymerase-specific fitting can also be fragile when extended to modified residues, nucleotide analogs, altered temperatures, streaming inputs, cross-platform analysis, or large-scale atlas generation. Additionally, some existing methods do not cleanly separate absolute timing behavior from normalized pause-pattern behavior, thereby making it harder to compare outputs across enzymes, conditions, or deployment architectures.

Accordingly, a need exists for improved computational methods that can accept a nucleic-acid sequence and one or more conditions and produce a deterministic, interpretable, position-resolved pause metric with little or no dependence on sequence-specific fitting. A need further exists for systems that can scale from short constructs to full genomic regions, from batch processing to real-time streaming, and from local execution to cloud and distributed embodiments, while preserving reproducibility and preserving support for modified residues, alternate enzyme classes, comparative condition analysis, and downstream biological use cases. The technical problem addressed by the present invention is how to compute, from sequence information and one or more operating conditions, a robust and position-resolved pause representation for a polymerase or other processive molecular machine in a manner that is deterministic, computationally efficient, physically interpretable, and broadly extensible. More specifically, there is a need for a method that maps a nucleic-acid sequence to a per-position kinetic barrier representation and derives from that representation one or more outputs such as per-position forward rate, dwell time, normalized pause density, ranked pause sites, clustered pause regions, or comparative pause metrics.

There is also a need for a method that avoids reliance on sequence-specific statistical training, repeated iterative fitting, or opaque parameter sets, while remaining compatible with multiple enzymes, multiple temperatures, modified nucleotides, quality-control workflows, therapeutic design workflows, and genome-scale or transcriptome-scale computation. There is a further need for a system that can be implemented in a deterministic software library, a command-line tool, an application programming interface, a batch-processing engine, a streaming analysis engine, an atlas-generation pipeline, or a combination thereof, while maintaining reproducibility and versionable computation.

In certain embodiments, the present invention employs a deterministic coherence-scale barrier quantity referred to herein as E_coh. In preferred embodiments, E_coh is derived from a Recognition Science framework in which a recognition composition law is expressed as: J(xy)+J(x/y)=2J(x)J(y)+2J(x)+2J(y) and a corresponding cost functional is expressed as: J(x)=0.5*(x+1/x)−1.

In illustrative embodiments, the recognition framework further yields: phi=(1+sqrt(5))/2. In some embodiments, a coherence quantum is expressed in RS-native dimensionless form as: E_coh=phi\hat{ }(−5). In some embodiments, an SI-energy reporting form of the coherence quantum is obtained using a selected unit convention, calibration, or reporting seam, and may be represented using a derived chain that includes: X_opt=phi/pi and E_coh=E_Planck*X_opt\hat{ }101, without limitation as to explanatory pathway, provided that the resulting coherence-scale barrier quantity used by the disclosed computation is explicitly defined and deterministically specified.

In illustrative SI-based embodiments, E_coh is approximately 0.090 eV. In preferred embodiments of the present invention, E_coh is used as a deterministic quantized barrier unit for sequence-dependent translocation, pause behavior, or related kinetic variation along a nucleic-acid sequence. The invention is not limited to any particular explanatory presentation, proof pathway, theoretical derivation, or provenance of E_coh, provided that the disclosed computation uses (a) the described coherence-scale barrier quantity E_coh or (b) an equivalent coherence-scale barrier quantity that is explicitly defined in the specification and is used in the disclosed deterministic sequence-to-barrier-to-rate/dwell/pause computation. In some embodiments, E_coh is representable in more than one mathematically equivalent form, including a normalized or dimensionless form derived from phi and a corresponding SI-energy form obtained by applying a selected unit convention, calibration, or reporting seam. Accordingly, E_coh may be supplied as a predefined constant, a version-controlled constants-bundle value, a computed value derived from a disclosed chain, or another deterministically specified coherence-scale parameter, without limiting the scope of the disclosed methods and systems.

Without being limited to any single mechanistic theory, the disclosed ladder-based framework treats local residue-dependent kinetic variation as a quantized barrier structure in which different residues, residue classes, local contexts, or modifications correspond to different effective barrier levels. In some embodiments, E_coh corresponds to a biological coherence-scale energy relevant to hydrogen-bond-mediated recognition, base-dependent interactions, stacking-related contributions, or other local molecular features that affect processive advancement of a polymerase or related machine. In some embodiments, the residue ladder assignment reflects a deterministic quantization of local base-dependent barrier structure, thereby enabling a sequence to be transformed into a position-resolved barrier representation from which rate, dwell, and pause metrics can be computed directly.

The present invention therefore provides a practical engineering framework in which sequence symbols are deterministically mapped into barrier indices, effective barrier indices, or related position-resolved representations, and those representations are used to compute one or more pause-related outputs without requiring machine-learning training, organism-specific fitting, or iterative statistical optimization. This framework addresses the shortcomings of prior approaches and provides an extensible basis for deterministic pause mapping across a range of biological, computational, analytical, and commercial applications.

### SUMMARY OF THE INVENTION

The present invention provides computer-implemented methods, systems, software, and non-transitory computer-readable media for deterministic mapping of polymerase pause behavior from a nucleic-acid sequence. In preferred embodiments, the invention receives a nucleic-acid sequence and one or more operating conditions, including temperature, and computes a position-resolved pause representation by assigning a barrier-related ladder index to each sequence position, calculating a corresponding forward rate and dwell time for each position, and generating one or more pause metrics for output, storage, visualization, ranking, annotation, or downstream analysis. In preferred embodiments, the invention performs such computation without sequence-specific machine-learning training, without iterative statistical fitting, and without organism-specific parameter tuning beyond one or more optionally selected enzyme or assay profiles.

In one aspect, the invention provides a method in which a nucleic-acid sequence is received, a ladder index n(i) is assigned to each position i, and one or more kinetic quantities are computed as a function of the ladder index, a coherence-scale barrier quantity E_coh, Boltzmann's constant k_B, temperature T, and an attempt-frequency term nu0 associated with an enzyme profile, assay profile, or operating condition. In preferred embodiments, the method computes an effective ladder index n_eff(i), a per-position forward rate k(i), a per-position dwell time tau(i), and a normalized pause metric sigma(i). In representative embodiments, the calculations are expressed in plain-text form as: n_eff(i)=n(i)+delta_nn(i)+delta_mod(i)+delta_ctx(i) k(i; p, T)=nu0(p)*exp(−(n_eff(i)*E_coh)/(k_B*T)) tau(i; p, T)=1/k(i; p, T) sigma(i; p, T)=tau(i; p, T)/sum_{j=1 to L} tau(j; p, T) where p identifies an enzyme profile, profile class, or operating mode, and L is the sequence length.

In another aspect, the invention provides a deterministic sequence-to-barrier mapping in which residues are transformed into a position-resolved barrier representation that can be computed using integer-valued ladder assignments, quantized correction terms, lookup tables, deterministic functions, or combinations thereof. In preferred embodiments, the core residue mapping assigns a respective ladder value to each of adenine, thymine, uracil, guanine, and cytosine. In further embodiments, one or more correction terms are applied to generate an effective ladder index n_eff(i), including nearest-neighbor corrections, local-context corrections, higher-order sequence-context corrections, modified-residue corrections, annotation-based corrections, or external deterministic corrections. In some embodiments, the correction terms are quantized in integer, half-integer, or other discrete multiples associated with the disclosed barrier framework.

In another aspect, the invention provides multi-output pause analysis rather than a single fixed output type. In some embodiments, the output includes one or more of a core ladder-index vector, an effective ladder-index vector, a per-position forward-rate vector, a per-position dwell-time vector, a normalized pause-density vector, a ranked pause-site list, a clustered pause-region list, a windowed pause score, a cumulative or regional pause burden, a comparative differential map, an annotation-linked report, or a visual output. In some embodiments, the normalized pause metric is used as a preferred representation of pause pattern because it preserves positional relative weighting while optionally removing dependence on any constant attempt-frequency scaling across positions.

In another aspect, the invention provides a profile-based computational architecture compatible with multiple processive molecular machines. In preferred embodiments, the invention is used for RNA polymerase pause mapping. In additional embodiments, the invention is applicable to DNA polymerases, reverse transcriptases, ribosome or codon-based translation embodiments, and other processive molecular machines that read a biological sequence and exhibit sequence-dependent translocation or pausing behavior. In some embodiments, the profile selection is made manually by a user. In other embodiments, the profile is selected automatically based on metadata, assay type, instrument type, target molecule type, or another input condition. In still further embodiments, a plurality of profiles may be applied to the same sequence to generate comparative results.

In another aspect, the invention provides deterministic implementations that are training-free, fitting-free or substantially free of sequence-specific fitting, reproducible, and versionable. In some embodiments, the computation is implemented as a pure functional pipeline that yields the same output for the same input under a fixed constants bundle and algorithm version. In some embodiments, the computation is configured for bit-identical output across deployments. In other embodiments, the computation is configured for numerical equivalence within a defined tolerance while preserving deterministic algorithmic structure. In some embodiments, hashes, checksums, version identifiers, constants manifests, verification certificates, or combinations thereof are stored or output to support auditability and regression testing.

In another aspect, the invention provides local and distributed software embodiments. In some embodiments, the invention is implemented as a local software library. In some embodiments, the invention is implemented as a command-line tool. In some embodiments, the invention is implemented as a web service, application programming interface, REST service, gRPC service, workflow component, notebook-accessible engine, embedded instrument module, or cloud analysis service. In some embodiments, the invention supports batch processing of multiple sequences, conditions, or enzyme profiles. In some embodiments, the invention supports streaming analysis in which sequence symbols or base calls are received incrementally and a rolling or partial pause profile is updated in real time. In some embodiments, the invention supports atlas generation in which pause-related outputs are precomputed for long templates, transcripts, genomes, transcriptomes, libraries, or reference collections and later served by coordinate-based or record-based query.

In another aspect, the invention provides comparative and perturbation-oriented embodiments. In some embodiments, the invention compares an unmodified sequence with a modified sequence. In some embodiments, the invention compares a baseline sequence with a sequence containing one or more modified residues, analogs, lesions, epigenetic marks, or therapeutic substitutions. In some embodiments, the invention compares outputs obtained under different temperatures, enzyme profiles, assay conditions, or chemical perturbations. In some embodiments, the invention computes a differential quantity such as delta_n(i), delta_tau(i), delta_sigma(i), or a ratio-based comparative metric. In some embodiments, the invention is used to identify modifications or perturbations that alter absolute timing while preserving normalized pause pattern, or that alter normalized pause pattern by changing local effective barrier structure.

In another aspect, the invention provides pause-site identification and regional analysis. In some embodiments, pause sites are identified by thresholding, percentile selection, top-N ranking, local-maximum detection, windowed scoring, clustering, merged-region calling, or annotation-guided aggregation. In some embodiments, the output includes one or more genomic coordinates, transcript coordinates, codon coordinates, gene-feature annotations, exon or intron annotations, promoter or terminator annotations, guide-seed annotations, or regulatory-region annotations associated with the computed pause metrics.

In another aspect, the invention provides downstream analysis and design use cases. In some embodiments, the invention is used for transcription analysis, promoter or terminator analysis, co-transcriptional folding analysis, comparative genomics, ribosome or codon optimization, guide RNA screening, therapeutic RNA quality control, vaccine or modified-RNA design review, instrument monitoring, single-molecule experiment support, or validation against experimental traces. In some embodiments, the invention is used to identify regions of elevated pause burden, conserved pause behavior, pause-sensitive design sites, or condition-dependent changes in sequence-dependent kinetic behavior.

In another aspect, the invention provides a system architecture in which deterministic sequence parsing, ladder assignment, optional correction, rate calculation, dwell-time computation, normalization, site calling, reporting, and storage are performed by discrete modules or by combined modules executing on one or more processors. In some embodiments, the modules are implemented in software. In some embodiments, one or more modules are accelerated using vector instructions, GPUs, distributed workers, or other hardware resources. In some embodiments, one or more outputs are stored in memory, persistent storage, indexed databases, compressed atlases, browser-compatible tracks, or machine-readable reports.

In another aspect, the invention provides optional formal verification and proof-checking embodiments. In some embodiments, one or more properties of the disclosed computation are verified, including bounded ladder-index behavior, positivity of rates, positivity of dwell times, normalization of a pause-density metric, monotonic ordering of rates or dwell times, deterministic execution, profile-invariance properties, or comparative identities. In some embodiments, such verification is implemented using Lean. In other embodiments, another formal verification, symbolic proof, model-checking, or theorem-proving environment may be used.

These and other aspects, embodiments, features, and advantages of the invention will be apparent from the following drawings, detailed description, and claims. The summary is provided to introduce selected concepts in simplified form and is not intended to identify every claimed feature, required implementation detail, or limiting aspect of the invention.

### BRIEF DESCRIPTION OF THE DRAWINGS

FIG. 1 is a block diagram of an overall Pause-Mapper system. The figure illustrates representative sequence input, computational, output, interface, and storage components that may be used in deterministic pause-mapping embodiments.

FIG. 2 is a flowchart of a high-level pause-mapping method. The figure illustrates receipt of sequence and condition data, computation of ladder-based kinetic quantities, and generation of one or more pause-related outputs.

FIG. 3 is a diagram of sequence input parsing and preprocessing. The figure illustrates representative handling of sequence records, canonicalization, ambiguity processing, coordinate preservation, and modified-residue parsing.

FIG. 4 is a diagram of residue-to-ladder-index mapping. The figure illustrates representative assignment of sequence residues to ladder values and generation of a corresponding position-resolved index representation.

FIG. 5 is a diagram of effective ladder-index correction. The figure illustrates representative incorporation of nearest-neighbor, context-dependent, modification-based, and annotation-based corrections to form an effective ladder index.

FIG. 6 is a diagram of rate, dwell-time, normalization, and pause-metric computation. The figure illustrates representative calculation of forward rates, dwell times, normalized pause values, and one or more derived pause metrics from the effective ladder index.

FIG. 7 is a diagram of pause-site calling, ranking, clustering, and output generation. The figure illustrates representative identification of pause sites or regions and generation of ranked, annotated, or exported outputs.

FIG. 8 is a diagram of a computing, API, storage, and non-transitory computer-readable medium embodiment. The figure illustrates representative processor, memory, interface, worker, storage, and machine-readable instruction components that may be used to implement the disclosed methods and systems.

### DETAILED DESCRIPTION OF THE INVENTION

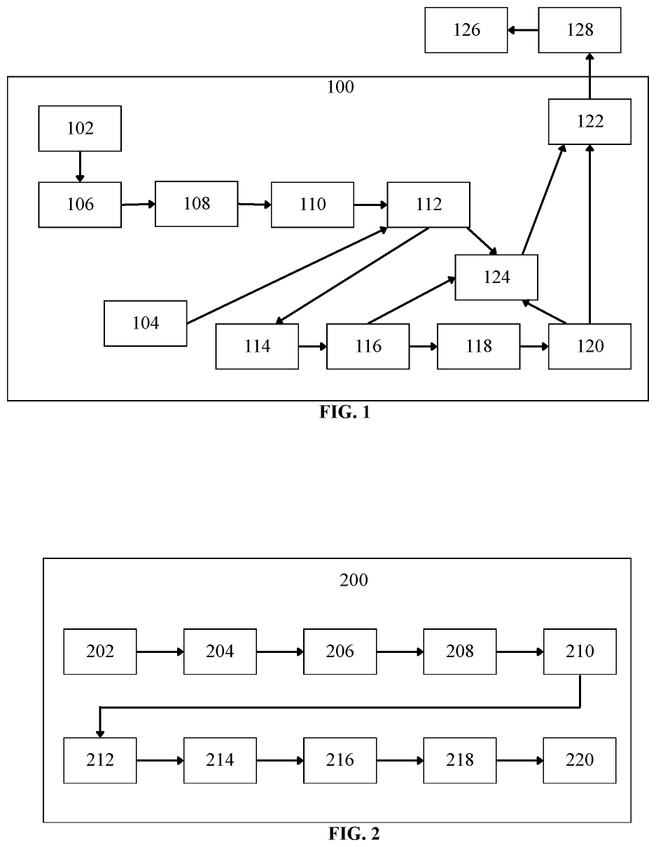

Referring now to the drawings, in which like reference numerals refer to like elements within a given figure series, FIG. 1 illustrates an overall Pause-Mapper system 100 configured to receive one or more sequence inputs from a sequence input source 102 together with one or more operating conditions, including a temperature input 104, and to process such inputs through a parser module 106, a ladder-index assignment engine 108, an effective-index correction engine 110, a rate calculation engine 112, a dwell-time engine 114, a normalization engine 116, and a pause-site calling engine 118 in order to produce one or more outputs by way of an output and report module 120. In some embodiments, the system 100 further communicates through an API and service interface 122, stores data or computed results in a data store 124, exchanges information with a client device 126, and operates across or through a network 128. Although particular functional blocks are illustrated in FIG. 1, the disclosed invention is not limited to the illustrated partitioning of functions, and in various embodiments one or more illustrated modules may be combined, separated, reordered, omitted, virtualized, distributed, accelerated, or otherwise implemented in hardware, software, firmware, or any suitable combination thereof.

As used herein, a “sequence” generally refers to an ordered set of biological symbols, residues, tokens, codons, or other machine-readable units that may be processed according to the disclosed methods. A “nucleic-acid sequence” refers to a DNA sequence, an RNA sequence, a DNA/RNA hybrid representation, a sequence containing canonical or non-canonical residues, a modified-residue sequence, a fragment thereof, a region thereof, a read therefrom, or any representation from which ordered residue positions may be determined. A “residue” refers to a base, nucleotide, analog, modified nucleotide, ambiguity-coded symbol, codon component, or another position-associated sequence unit. A “base position” refers to a location within a sequence, whether indexed by zero-based indexing, one-based indexing, coordinate indexing, genomic indexing, transcript indexing, codon indexing, or another indexing convention. A “codon” refers to a triplet or other grouped translation-related unit derived from a sequence and may, in some embodiments, serve as a higher-order processing unit for pause-related analysis.

As used herein, a “polymerase” refers broadly to an RNA polymerase, DNA polymerase, reverse transcriptase, or another processive molecular machine that advances along a nucleic-acid template or related sequence while exhibiting sequence-dependent kinetic behavior. A “processive molecular machine” refers more broadly to any molecular complex, biological complex, synthetic system, or modeled sequence-reading system that moves along, reads, synthesizes from, translates from, or otherwise interacts sequentially with a biological or encoded sequence and exhibits position-dependent advancement, dwell, pause, stall, or related kinetic variation. A “ladder index” refers to a deterministic value associated with a residue or position and representative of a barrier level, barrier rung, quantized energetic contribution, or other position-resolved kinetic descriptor used in the disclosed computation. An “effective ladder index” refers to a ladder index after one or more deterministic corrections, context terms, modification terms, annotation terms, or other disclosed adjustments have been incorporated.

In some embodiments, all disclosed machine types (including RNA polymerases, DNA polymerases, reverse transcriptases, and ribosome-associated translation systems) share a common inventive concept comprising a deterministic pipeline that (i) maps sequence symbols at ordered positions to a quantized barrier representation via a core ladder index and an effective ladder index, (ii) computes one or more per-position kinetic quantities as a deterministic function of the effective ladder index and one or more operating conditions including temperature, and (iii) derives one or more pause metrics from the per-position kinetic quantities for output, ranking, clustering, streaming updates, comparative analysis, or atlas generation. Machine-specific embodiments differ primarily by profile selection and optional deterministic correction policies applied to form the effective ladder index.

As used herein, a “coherence quantum” refers to a coherence-scale barrier quantity, including E_coh, used in determining a rate, dwell, pause, barrier, or related position-dependent calculation. An “attempt frequency” refers to a frequency term, profile term, profile bundle term, registry value, or analogous factor associated with an enzyme, assay, instrument, condition, or operating mode and used in determining one or more rate-related values. A “pause metric” refers to any position-resolved or region-resolved output indicative of sequence-dependent slowing, pausing, stalling, dwell weighting, cluster burden, local score, normalized pause density, rank, threshold exceedance, differential behavior, or other pause-related characteristic. A “pause density” refers to a normalized position-wise distribution derived from one or more dwell-like quantities, residence-like quantities, or related kinetic quantities. A “pause site” refers to a position, interval, cluster, region, codon, grouped residue set, or other identified portion of a sequence that satisfies one or more pause-related criteria.

As used herein, a “cluster” refers to a grouped set of nearby positions, sites, intervals, or regions that are merged, associated, ranked, or otherwise handled together based on one or more pause metrics, thresholds, densities, distances, or scoring rules. A “modified residue” refers to a chemically modified nucleotide, analog, synthetic base, damaged residue, tagged residue, substituted residue, labeled residue, therapeutic residue, or another non-canonical or specially designated sequence symbol. An “annotation input” refers to any metadata, feature data, coordinate annotation, mask, lookup source, rule source, user-supplied record, experiment-derived record, or other externally supplied information that can be used in parsing, correction, filtering, comparison, or output generation. A “stream” refers to an incrementally received sequence, partial sequence, rolling base-call sequence, live instrument output, chunked input, or other temporally progressive input. An “atlas” refers to a precomputed or partially precomputed collection of pause-related outputs, scores, vectors, summaries, regions, or indexed records for one or more sequences, templates, genomes, transcriptomes, sequence libraries, or design collections. A computation is “deterministic” when, under a defined input set, constants set, profile selection, algorithm version, and implementation policy, the computation produces the same substantive result without stochastic training, random sampling, or iterative fitting dependence.

In some embodiments, the disclosed computation provides a technical improvement in deterministic sequence analytics and bioinformatics computing by (i) transforming an input sequence into a position-resolved barrier representation using a fixed ladder mapping and quantized correction policy, (ii) computing position-resolved rates, dwell times, and normalized pause metrics in linear time with respect to sequence length using a fixed constants bundle and profile selection, and (iii) producing reproducible, versionable outputs suitable for batch, streaming, and atlas-generation deployments without training, iterative fitting, or convergence-dependent variability. In some embodiments, the system further improves computer functionality by enabling rolling updates for streamed sequence symbols, by caching precomputed pause atlases for coordinate-addressable query, and by emitting provenance data including algorithm-version identifiers, constants-bundle identifiers, and checksums for regression testing.

For example, in some embodiments, a pause-mapping request is received through an application programming interface, a compute worker executes the deterministic pipeline under a fixed numerical policy and constants bundle, and results are stored in an indexed result store for later coordinate-based retrieval, thereby reducing recomputation and improving query latency for repeated analyses over large templates or libraries. In some embodiments, the system emits a manifest comprising a profile identifier, constants-bundle identifier, algorithm-version identifier, and checksum for each output payload to support auditability and regression testing across deployments.

Unless the context clearly indicates otherwise, singular terms include the plural and plural terms include the singular. The terms “comprising,” “including,” “having,” “containing,” and analogous open-ended terms are intended to be inclusive rather than exclusive. References to “one embodiment,” “some embodiments,” “preferred embodiments,” “illustrative embodiments,” or similar language indicate that the described feature, structure, or characteristic may be included in at least one embodiment of the invention, but need not be included in every embodiment. Steps described herein may be performed in the order presented, in a different order, in parallel, in partially overlapping order, or in another suitable order unless a particular order is explicitly required. Arithmetic may be implemented in direct form, transformed form, normalized form, scaled form, log-space form, lookup-based form, vectorized form, hardware-accelerated form, approximate numerically equivalent form, or any other suitable computational form consistent with the disclosed functions. Arrays, vectors, lists, matrices, maps, tables, streams, tuples, records, objects, frames, and other data structures may be used interchangeably where appropriate to store or represent disclosed values.

In some embodiments, determinism is enforced by an explicit numerical policy comprising: (i) use of a fixed floating-point representation for rate, dwell, and normalization calculations; (ii) use of a fixed evaluation order for reductions used in normalization and derived metrics; (iii) a fixed constants bundle including E_coh and k_B; (iv) a fixed profile bundle including nu0 and any correction-policy selections; and (v) emission of a version identifier and checksum for each output. In some embodiments, the numerical policy specifies IEEE-754 double-precision arithmetic for exponentials and divisions, and specifies one or more deterministic summation strategies for normalization, including blockwise reduction or compensated summation, such that repeated execution under a fixed policy yields identical or tolerance-bounded equivalent outputs across deployments.

Referring now to FIG. 2, a pause-mapping method 200 is shown in representative form. In some embodiments, the method begins by receiving a sequence at 202 and receiving a temperature and, optionally, an enzyme identity, profile identifier, assay condition, or related profile selector at 204. The method may then canonicalize and validate the received sequence at 206, assign core ladder indices at 208, compute effective ladder indices at 210, compute rates at 212, compute dwell times at 214, compute a normalized pause metric at 216, identify pause sites or regions at 218, and output one or more results at 220. The illustrated order is representative only. In some embodiments, one or more operations may be merged, repeated, omitted, conditionally invoked, streamed, parallelized, or performed incrementally on a rolling basis.

In representative embodiments, a sequence S has length L and contains ordered residues b_1 through b_L. A temperature T may be supplied as a scalar condition, a profile-dependent condition, a time-varying condition, a position-varying condition, or another suitable operating parameter. A profile p may identify an enzyme, enzyme class, assay mode, operating bundle, registry entry, or comparative condition. A core residue index n(i) may be assigned to each position i, and an effective index n_eff(i) may be determined as a function of the core index and one or more deterministic correction terms. In representative embodiments, the computation may be expressed as: n_eff(i)=n(i)+delta_nn(i)+delta_mod(i)+delta_ctx(i)+delta_ext(i) where delta_nn(i) represents a nearest-neighbor or local adjacency correction, delta_mod(i) represents a modified-residue correction, delta_ctx(i) represents a context-related correction, and delta_ext(i) represents an annotation-based, profile-based, or other externally supplied deterministic correction. In some embodiments, one or more of these correction terms may be omitted, set to zero, selected from a lookup table, determined by a deterministic rule, quantized to an integer or half-integer value, or otherwise generated according to a disclosed implementation policy.

As used in the representative equations herein: i denotes an ordered position in the sequence; j denotes a summation index over positions; L denotes a sequence length or number of analyzed units; p denotes a selected profile associated with a processive molecular machine; n(i) denotes a core ladder index for position i; n_eff(i) denotes an effective ladder index for position i; delta_nn(i) denotes a nearest-neighbor correction term; delta_mod(i) denotes a modified-residue correction term; delta_ctx(i) denotes a context-related correction term; delta_ext(i) denotes an external-annotation or externally supplied deterministic correction term; k(i; p, T) denotes a per-position forward rate; tau(i; p, T) denotes a per-position dwell time; sigma(i; p, T) denotes a normalized pause metric; nu0(p) denotes an attempt-frequency term associated with profile p; E_coh denotes a coherence-scale barrier quantity; k_B denotes Boltzmann's constant; and T denotes an absolute temperature.

In representative embodiments, the rate calculation engine 112 of FIG. 1 and the computation pathway illustrated in FIG. 2 calculate a per-position forward rate according to: k(i; p, T)=nu0(p)*exp(−(n_eff(i)*E_coh)/(k_B*T)) where nu0(p) is an attempt-frequency term associated with profile p, E_coh is the disclosed coherence quantum, and k_B is Boltzmann's constant. A per-position dwell time may then be determined according to: tau(i; p, T)=1/k(i; p, T) and a normalized pause metric may be determined according to: sigma(i; p, T)=tau(i; p, T)/sum_{j=1 to L} tau(j; p, T).

In some embodiments, the system outputs one or more of n(i), n_eff(i), k(i), tau(i), sigma(i), a ranked site list, a cluster list, a differential score, a ratio score, a windowed score, a burden score, a report, a visualization, or an exported machine-readable record. In some embodiments, the normalized pause metric may reduce or eliminate dependence on a constant profile-wide scaling of nu0 across positions, thereby supporting comparison of pause patterns across different enzyme profiles or assay conditions.

The disclosed invention is not limited to the specific architecture shown in FIGS. 1 and 2, nor to a single species of nucleic acid, polymerase, profile type, output type, deployment mode, or theory presentation. Rather, the disclosed invention encompasses deterministic implementations in which a sequence is transformed into a position-resolved barrier representation and one or more pause-related outputs are computed therefrom in a reproducible and extensible manner suitable for local, remote, batch, streaming, comparative, atlas-scale, or verification-enabled use.

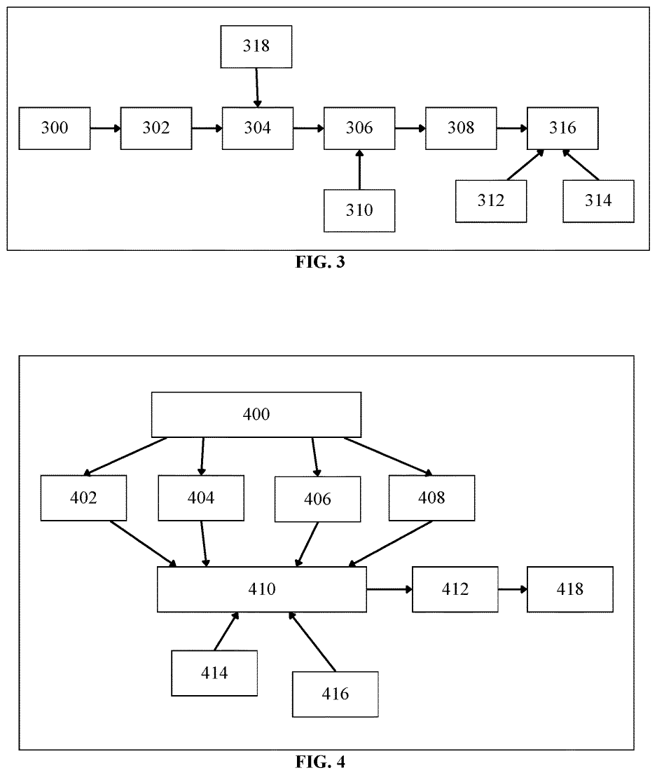

Referring now to FIG. 3, a representative input parsing and preprocessing embodiment is shown. In some embodiments, the disclosed system receives a sequence record 300 that contains, points to, or otherwise provides access to a residue string 302 together with optional contextual data, including one or more identifiers, coordinate ranges, strand indicators, source labels, profile selectors, assay descriptors, sample descriptors, modification annotations, or other metadata. The sequence record 300 may be received from a file, a memory object, a database record, a streamed message, an instrument output, a web request, an application programming interface call, a command-line argument, a workflow engine, or another suitable source. In some embodiments, the sequence record 300 corresponds to a single sequence. In other embodiments, the sequence record 300 corresponds to a batch, set, collection, stream, or grouped input containing multiple sequences, multiple regions, multiple conditions, or combinations thereof.

The residue string 302 may comprise canonical DNA symbols, canonical RNA symbols, non-canonical residue symbols, ambiguity-coded symbols, modified-residue indicators, codon-grouped symbols, mixed-case symbols, whitespace-containing strings, line-broken strings, formatted sequence content, or other machine-readable representations from which an ordered residue sequence can be determined. In some embodiments, the residue string 302 is supplied in plain text form. In some embodiments, the residue string 302 is extracted from FASTA content, FASTQ content, GenBank content, coordinate-addressable reference content, transcript records, sequence-library entries, or another sequence-containing source. In some embodiments, the residue string 302 is supplied incrementally as a stream of base calls, rolling sequence chunks, or partial reads. In some embodiments, the sequence record 300 includes a plurality of residue strings 302 corresponding to distinct candidates, guides, constructs, amplicons, transcripts, genomic regions, or comparative conditions.

A format detector 304 may evaluate the incoming sequence record 300 to determine how the residue string 302 and related fields are encoded. In some embodiments, the format detector 304 identifies whether the incoming content is plain text, FASTA, FASTQ, GenBank, delimited tabular input, structured object input, a serialized API payload, a binary protocol payload, or another recognizable format. In some embodiments, the format detector 304 identifies header fields, quality lines, annotation sections, coordinate indicators, strand indicators, sequence identifiers, modification tags, or other syntactic markers. In some embodiments, the format detector 304 is implemented as a parser-selection stage that dispatches the sequence record 300 to one of a plurality of parsing policies. In other embodiments, the format detector 304 may be omitted and the format may be supplied explicitly by a caller, stored in metadata, or inferred from context.

After format identification or format assumption, the residue string 302 may be passed to a canonicalizer 306. In some embodiments, the canonicalizer 306 removes or ignores whitespace, line breaks, separators, formatting marks, comments, or non-sequence delimiters. In some embodiments, the canonicalizer 306 normalizes case, such as by converting lowercase residue symbols to uppercase residue symbols. In some embodiments, the canonicalizer 306 converts equivalent sequence expressions to a canonical internal form. For example, thymine and uracil may be preserved distinctly, unified under a policy rule, or converted depending on whether the input is treated as DNA, RNA, a hybrid, or a codon-derived representation. In some embodiments, the canonicalizer 306 enforces a selected alphabet policy, a residue validation policy, a modification-tag preservation policy, an ambiguity policy, or a streaming-consistency policy. In some embodiments, the canonicalizer 306 outputs a standardized intermediate representation suitable for downstream ladder assignment and correction calculations.

In some embodiments, the sequence undergoes ambiguity handling by an ambiguity-code handler 308. The ambiguity-code handler 308 may process one or more ambiguous symbols, such as symbols representing multiple possible bases, uncertain calls, mixed populations, low-confidence calls, masked positions, or unspecified residues. In some embodiments, the ambiguity-code handler 308 replaces each ambiguous symbol with an expected ladder value computed over a candidate residue set. For example, in a representative embodiment, an ambiguity-coded position B_amb(i) may be associated with a set R(B_amb(i)) of possible residues, and a corresponding ladder value may be computed as: n(B_amb(i))=sum_{r in R(B_amb(i))} P(r|B_amb(i))*n(r) where P(r|B_amb(i)) is a probability, weight, or policy weight associated with residue r under the ambiguity designation. In some embodiments, the ambiguity-code handler 308 uses an expectation rule. In other embodiments, the ambiguity-code handler 308 may use a deterministic minimum rule, maximum rule, midpoint rule, weighted rule, lookup-table rule, profile-conditioned rule, context-conditioned rule, or other policy-based assignment. In some embodiments, ambiguity handling is deferred until a later stage, including the effective-index stage described elsewhere herein. In some embodiments, ambiguous positions are retained as structured objects rather than being collapsed immediately to a scalar ladder value.

In some embodiments, a strand selector 310 is provided to select, infer, or transform strand orientation. The strand selector 310 may preserve the residue order as received, reverse the residue order, compute a reverse complement, compute a complement without reversal, convert between template and coding strand conventions, or otherwise transform the sequence according to a user-specified, metadata-specified, or policy-specified orientation rule. In some embodiments, the strand selector 310 is used where the disclosed pause mapping is intended to follow a particular transcriptional or translational direction. In some embodiments, the strand selector 310 may be omitted where sequence direction is fixed by the input source. In some embodiments, a plurality of strand or orientation interpretations may be evaluated to generate comparative outputs.

In some embodiments, a coordinate mapper 312 preserves, converts, or generates positional mappings associated with the processed sequence. The coordinate mapper 312 may associate each internal sequence position with a genomic coordinate, transcript coordinate, feature-relative coordinate, codon coordinate, read coordinate, local window coordinate, batch index, or another positional identifier. In some embodiments, the coordinate mapper 312 preserves offsets from an original sequence record 300 even when preprocessing removes line breaks, headers, separators, quality strings, modification tags, or non-residue formatting. In some embodiments, the coordinate mapper 312 supports coordinate extraction from a larger reference sequence, such as a chromosome, transcript, plasmid, viral genome, synthetic construct, or sequence library entry.

In some embodiments, the coordinate mapper 312 supports mapping pause-related outputs back onto one or more external annotation systems, browser tracks, reporting tables, guide-design systems, validation traces, or downstream software tools.

In some embodiments, the input parsing and preprocessing stage includes a modified-residue parser 314. The modified-residue parser 314 may identify one or more non-canonical residues, analogs, chemically modified bases, tagged residues, synthetic residues, therapeutic substitutions, lesions, adducts, epigenetic marks, or annotation-linked symbols associated with one or more positions. In some embodiments, the modified-residue parser 314 reads explicit symbols embedded directly in the residue string 302. In some embodiments, the modified-residue parser 314 reads tags, sidecar records, annotation fields, structured metadata, or coordinate-linked modification files associated with the sequence record 300. In some embodiments, the modified-residue parser 314 generates a per-position modification representation that can later be used by the effective-index correction engine 110 of FIG. 1 or by another downstream computation stage. In some embodiments, the modified-residue parser 314 preserves the original canonical residue identity and stores the modification as a layered annotation. In other embodiments, the modified-residue parser 314 maps the modified residue directly into a canonicalized internal symbol plus one or more associated correction indicators. Following parsing, canonicalization, ambiguity handling, strand handling, coordinate handling, and modification parsing, the sequence may be represented as a validated sequence array 316. The validated sequence array 316 may comprise a contiguous array, vector, list, stream buffer, chunked array, sparse representation, token sequence, record set, or other data structure storing the ordered sequence in a machine-readable form suitable for downstream processing. In some embodiments, the validated sequence array 316 contains only canonical residues. In some embodiments, the validated sequence array 316 contains canonical residues plus attached ambiguity information, modification information, coordinate information, or profile-linked information. In some embodiments, the validated sequence array 316 is stored in a memory-efficient format suitable for batch, genome-scale, or streaming implementations. In some embodiments, the validated sequence array 316 is generated incrementally so that the disclosed system can operate on a rolling prefix or partial sequence prior to receiving the entire input.

In some embodiments, metadata associated with the processed input is stored or represented as a metadata record 318. The metadata record 318 may include one or more identifiers, accession numbers, source fields, enzyme-profile selectors, instrument fields, timestamps, coordinate offsets, sequence lengths, alphabet indicators, canonicalization policies, ambiguity-handling policies, modification-handling policies, hash values, checksum values, algorithm versions, constants-bundle versions, reporting options, or output preferences. In some embodiments, the metadata record 318 is used to drive profile selection, output formatting, reproducibility controls, audit logging, or downstream annotation linkage. In some embodiments, the metadata record 318 is passed together with the validated sequence array 316 to downstream computational stages. In some embodiments, the metadata record 318 may also include user-provided or system-generated fields indicating whether the input is intended for RNA polymerase analysis, DNA polymerase analysis, reverse transcriptase analysis, codon-based analysis, guide-screening analysis, quality-control analysis, or another disclosed use case.

The output of the FIG. 3 preprocessing embodiment may therefore include the validated sequence array 316 and the metadata record 318, either of which may be passed to the ladder-index assignment engine 108 of FIG. 1 and to subsequent stages of the method 200 shown in FIG. 2. In some embodiments, validation includes confirming that the processed sequence satisfies one or more acceptance rules, including symbol validity, coordinate consistency, modification-tag consistency, stream continuity, non-empty content, minimum-length requirements, maximum-length requirements, or profile compatibility. In some embodiments, invalid input may be rejected, corrected, masked, partially accepted, flagged, or transformed according to a specified policy.

The FIG. 3 embodiment supports a broad range of practical inputs and use cases. By accepting plain-text sequences, formatted records, streamed base calls, coordinate-derived subsequences, modified-residue annotations, ambiguity-coded positions, and metadata-linked records, the disclosed system can be used in local software, batch-processing, cloud-service, real-time instrument, atlas-generation, therapeutic-design, and comparative-analysis implementations without altering the core deterministic computational framework. The preprocessing pathway further preserves sufficient positional, symbolic, and contextual information to support later residue-to-ladder assignment, effective-index correction, pause-site identification, annotation-linked reporting, and differential comparison without requiring sequence-specific statistical training or iterative fitting.

Referring now to FIG. 4, a representative residue-to-ladder-index mapping embodiment is shown. In some embodiments, the disclosed system transforms the validated sequence array 316 into a position-resolved ladder representation by applying a mapping table 400 to each residue position. The mapping table 400 may be implemented as a static lookup structure, a deterministic rule set, a compiled table, a branch-based routine, a vectorized instruction path, a hardware-supported mapping mechanism, or another suitable implementation that deterministically associates one or more sequence symbols with one or more ladder values. In preferred embodiments, the mapping performed in FIG. 4 supplies the core index n(i) for each sequence position i and thereby generates the primary barrier representation used by later computation stages, including the effective-index correction engine 110, the rate calculation engine 112, the dwell-time engine 114, and the normalization engine 116 of FIG. 1.

In preferred embodiments, the mapping table 400 assigns a respective ladder value to each canonical base according to a deterministic residue-to-index rule. In an illustrative implementation, adenine is processed through an adenine branch 402, thymine and uracil are processed through a thymine and uracil branch 404, guanine is processed through a guanine branch 406, and cytosine is processed through a cytosine branch 408. In preferred embodiments, the corresponding core ladder assignments are expressed as:
n(A)=0
n(T)=1
n(U)=1
n(G)=2
n(C)=3.

In some embodiments, the ladder values are integer-valued and represent quantized barrier levels, quantized coherence-related energetic contributions, or another deterministic rung structure suitable for later kinetic computation. In some embodiments, adenine corresponds to the lowest core rung, thymine and uracil correspond to a next rung, guanine corresponds to a higher rung, and cytosine corresponds to the highest core rung among the canonical single-residue assignments. The invention is not limited to any single explanatory mechanism for why these assignments are useful, but in some embodiments the ordering reflects one or more of hydrogen-bond contribution, residue-class contribution, stacking-related contribution, local recognition-stable interaction behavior, or another deterministic barrier-related interpretation consistent with the disclosed framework.

In some embodiments, the lookup engine 410 applies the mapping table 400 position by position across the processed sequence. The lookup engine 410 may operate on a single residue at a time, on a vector of residues, on a chunk of residues, on a streaming buffer, on a compressed representation, or on another internal data structure. In some embodiments, the lookup engine 410 outputs a ladder-index array 412 comprising a position-resolved sequence of core ladder values. The ladder-index array 412 may be stored as an integer array, floating-point array, quantized array, packed array, sparse representation, streaming representation, segmented representation, or another suitable machine-readable form. In some embodiments, the ladder-index array 412 is represented as: N=[n(1), n(2), . . . , n(L)] where L is the sequence length. In some embodiments, the ladder-index array 412 is retained in memory for immediate downstream use. In some embodiments, the ladder-index array 412 is persisted, streamed, hashed, versioned, or exported for later analysis.

In some embodiments, the residue-to-ladder-index mapping is performed using branch logic rather than an explicit table. For example, the adenine branch 402 may identify one or more adenine symbols and assign a core ladder value of zero, the thymine and uracil branch 404 may identify one or more thymine or uracil symbols and assign a core ladder value of one, the guanine branch 406 may identify one or more guanine symbols and assign a core ladder value of two, and the cytosine branch 408 may identify one or more cytosine symbols and assign a core ladder value of three. In some embodiments, such branch logic may be compiled into if-then logic, switch logic, finite-state logic, machine-code branch tables, SIMD-compatible masks, GPU kernels, or another deterministic processing structure. In some embodiments, the branch implementations and table implementations are functionally equivalent and interchangeable.

In some embodiments, the mapping process accommodates ambiguity-coded or policy-dependent symbols through an ambiguity expectation engine 414. The ambiguity expectation engine 414 may receive one or more positions previously marked by the ambiguity-code handler 308 of FIG. 3 and may compute a scalar ladder value, a vector-valued ladder representation, a bounded interval, or another deterministic representation for such positions. In some embodiments, the ambiguity expectation engine 414 uses an expectation formulation, such as: n(B_amb(i))=sum_{r in R(B_amb(i))} P(r|B_amb(i))*n(r) where R(B_amb(i)) is a candidate residue set and P(r|B_amb(i)) is a deterministic weight or probability associated with residue r under the selected ambiguity policy. In some embodiments, the output of the ambiguity expectation engine 414 may be non-integer, thereby producing a scalar intermediate value suitable for later effective-index processing. In other embodiments, ambiguity-coded positions are resolved through a deterministic fallback rule, a conservative rule, a permissive rule, a maximum-barrier rule, a minimum-barrier rule, a midpoint rule, or a selected candidate rule.

In some embodiments, a fallback rule 416 is used when an input symbol is unrecognized, partially recognized, masked, disallowed, profile-dependent, or otherwise not directly mapped by the canonical rules. The fallback rule 416 may reject the symbol, map the symbol to a masked state, map the symbol to an ambiguity state, map the symbol to a default ladder value, invoke a profile-specific conversion rule, invoke a modification parser output, invoke an external annotation mapping, or otherwise resolve the symbol according to a defined implementation policy. In some embodiments, the fallback rule 416 is selected to preserve deterministic behavior by avoiding stochastic selection, random imputation, or training-based inference. In some embodiments, the fallback rule 416 may be configurable at deployment time, compile time, runtime, or per-request time, provided that the selected policy remains explicit and reproducible for a given computation.

The output of the FIG. 4 embodiment is an index output 418 that may correspond to a complete ladder-index array 412, a partial ladder-index array 412, a rolling prefix representation, a chunk-local representation, or another core residue-to-index output suitable for subsequent processing. In some embodiments, the index output 418 is fed directly to the effective-index correction engine 110 of FIG. 1. In some embodiments, the index output 418 is also stored or exposed as an independent output because the core ladder pattern itself may be useful for analysis, visualization, comparison, quality review, debugging, verification, or downstream design purposes.

In preferred embodiments, the FIG. 4 mapping stage provides a deterministic and bounded residue-to-index transformation that can be executed in linear time with respect to sequence length. In some embodiments, the mapping stage operates in O(L) time and O(L) memory for a sequence of length L. In other embodiments, the memory footprint may be reduced using streaming, compression, windowed processing, chunked processing, or in-place transformation. In some embodiments, the mapping stage is implemented on one or more CPUs. In some embodiments, the mapping stage is implemented using vector instructions. In some embodiments, the mapping stage is accelerated using one or more GPUs, specialized accelerators, field-programmable gate arrays, or other hardware. In some embodiments, the mapping stage is distributed across multiple workers or shards for large sequence collections, atlas-generation workflows, or comparative batch analysis.

The FIG. 4 embodiment is not limited to a single type of sequence or a single type of processive machine. In some embodiments, the residue-to-ladder assignment is applied directly to DNA sequences. In some embodiments, the residue-to-ladder assignment is applied directly to RNA sequences. In some embodiments, the same core residue mapping is used as an input layer for codon-based analysis, reverse transcriptase analysis, DNA polymerase analysis, RNA polymerase analysis, or another disclosed embodiment. In some embodiments, the core ladder mapping remains fixed while later stages incorporate enzyme-specific corrections, context-dependent corrections, modification-dependent corrections, or output-dependent transformations. This separation between core residue mapping and later correction stages provides a robust disclosure structure because it allows the basic residue-to-index transform to remain simple, deterministic, and reusable while still supporting more elaborate embodiments elsewhere in the specification.

The FIG. 4 embodiment therefore establishes the core transformation by which an ordered sequence of residues is converted into an ordered sequence of ladder values that can be used as a deterministic barrier representation. This core representation may subsequently be refined, corrected, normalized, compared, or otherwise processed in later stages while preserving a clear computational lineage from the original sequence symbols to the resulting pause-related outputs.

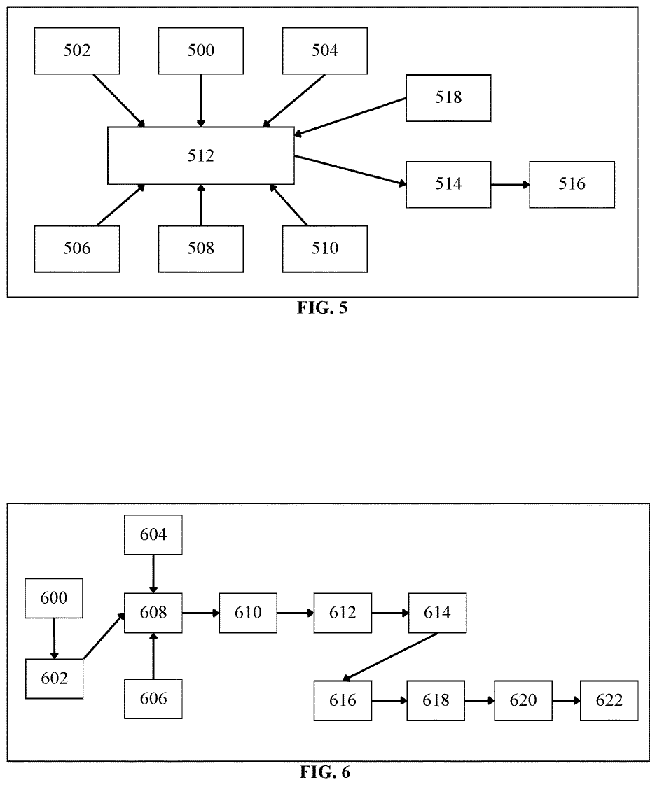

Referring now to FIG. 5, a representative effective ladder-index correction embodiment is shown. In some embodiments, the core ladder-index values generated from the residue-to-ladder mapping stage are refined to account for local sequence context, neighboring residues, modified residues, annotations, or other deterministic factors that may affect position-resolved barrier behavior. In preferred embodiments, a current residue position 500 is evaluated together with one or more neighboring or associated inputs to generate an effective ladder index 514 that is used in later rate, dwell, normalization, and pause-metric computations. The FIG. 5 embodiment therefore supports extension of the core mapping framework beyond single-residue assignments while preserving deterministic and quantized computation.

In some embodiments, the current residue position 500 is evaluated with respect to an upstream context 502 and a downstream context 504. The upstream context 502 may include one or more residues positioned before the current residue position 500, one or more already-parsed codon or motif units, one or more coordinate-linked annotations, or one or more previously computed state values. The downstream context 504 may include one or more residues positioned after the current residue position 500, one or more lookahead values, one or more motif indicators, one or more modification-linked indicators, or one or more profile-linked context indicators. In some embodiments, the upstream context 502 and downstream context 504 are each limited to one neighboring residue. In other embodiments, either or both may include a larger window, a codon-scale grouping, a motif-scale grouping, a regulatory feature grouping, or another selected context span.

In some embodiments, the disclosed system uses a nearest-neighbor correction store 506 to provide one or more deterministic corrections associated with adjacent residues. For example, a nearest-neighbor correction may be determined as a function of a pair of residues that includes the current residue position 500 and an adjacent residue in the upstream context 502 or downstream context 504. In representative embodiments, such a correction may be expressed as: n_eff(i)=n(i)+delta_nn(b_{i−1}, b_i) or, in another representative embodiment, as: n_eff(i)=n(i)+delta_left(b_{i−1}, b_i)+delta_right(b_i, b_{i+1}) where the delta terms are deterministic correction values associated with the neighboring residue identities and their arrangement. In some embodiments, the nearest-neighbor correction store 506 contains precomputed entries for all canonical adjacent residue combinations. In some embodiments, the nearest-neighbor correction store 506 contains entries that depend on direction, strand interpretation, enzyme profile, sequence class, or assay condition. In some embodiments, a nearest-neighbor correction is omitted by assigning the corresponding correction term to zero.

In some embodiments, higher-order context is represented by a higher-order correction store 508. The higher-order correction store 508 may provide correction values derived from triplets, codons, windows, motifs, local subsequences, grouped residue patterns, or another selected context size. In representative embodiments, a higher-order context correction may be expressed as: n_eff(i)=n(i)+delta_k(b_{i−k}, . . . , b_{i+k}) where delta_k is a deterministic correction associated with a context window extending from position i−k through position i+k. In some embodiments, the higher-order correction store 508 is implemented as a lookup table keyed by a context token, sequence motif, codon token, regulatory feature type, or hashed local subsequence. In some embodiments, the higher-order correction store 508 is implemented as a deterministic function rather than a literal table. In some embodiments, the higher-order correction store 508 is profile-specific, species-specific, construct-specific, or application-specific, provided that the selected policy remains explicit, versionable, and reproducible.

In some embodiments, modification-dependent correction is provided through a modification correction store 510. The modification correction store 510 may store or generate one or more correction values associated with a modified residue, a nucleotide analog, a synthetic residue, a lesion, an adduct, a methylated residue, pseudouridine, or another modified or annotated sequence component identified by the modified-residue parser 314 of FIG. 3. In representative embodiments, a modification correction may be expressed as: n_eff(i)=n(i)+delta_mod(m_i) where m_i denotes a modification state, modification label, modification class, or other modification-associated designation for position i. In some embodiments, the modification correction store 510 distinguishes between multiple classes of modified residues at the same canonical base identity. In some embodiments, the modification correction store 510 applies no correction where a position has no registered modification or where a selected profile does not use modification-aware processing.

In some embodiments, the current residue position 500 may also be influenced by an external annotation input 518. The external annotation input 518 may include one or more feature labels, regulatory annotations, experimentally derived marks, structural indicators, quality-control flags, therapy-design annotations, region masks, coding-frame indicators, known motif annotations, or other metadata associated with the position. In some embodiments, such external information contributes an additional deterministic correction, represented for example by delta_ext(i), thereby allowing the effective index to incorporate user-supplied or system-supplied annotation-dependent behavior without changing the core ladder assignment stage. In some embodiments, the external annotation input 518 is used only for downstream output and not for effective-index modification. In other embodiments, the external annotation input 518 directly participates in effective-index formation.

In preferred embodiments, one or more correction terms are combined by a quantization engine 512 to produce the effective ladder index 514. The quantization engine 512 may receive one or more inputs from the nearest-neighbor correction store 506, the higher-order correction store 508, the modification correction store 510, and the external annotation input 518 and may combine such terms according to a deterministic policy. In representative embodiments, the effective ladder index 514 may be expressed as: n_eff(i)=n(i)+delta_nn(i)+delta_mod(i)+delta_ctx(i)+delta_ext(i) where delta_ctx(i) may include one or more higher-order or bidirectional context terms. In some embodiments, each correction term may be an integer, a half-integer, a rational value, or another quantized multiple associated with the disclosed barrier framework. In some embodiments, one or more correction terms may be stored or computed in units corresponding directly or indirectly to E_coh. In some embodiments, the quantization engine 512 rounds, clamps, preserves, transforms, or otherwise normalizes incoming correction terms to satisfy a selected implementation policy. In some embodiments, the quantization engine 512 preserves a non-integer scalar value for direct use in later rate calculations. In some embodiments, the quantization engine 512 maps a computed value onto a discrete permitted set before output.

The effective ladder index 514 may therefore differ from the core ladder value n(i) while still maintaining a deterministic computational lineage from the original sequence. In some embodiments, the effective ladder index 514 captures subtle variation caused by local sequence arrangement, codon context, modification state, or annotation-linked state. In some embodiments, the effective ladder index 514 is stored as a scalar per position. In some embodiments, the effective ladder index 514 is stored as a vector or record with component terms for later interpretability, debugging, auditing, verification, or reporting. In some embodiments, the effective ladder index 514 may be associated with a confidence, provenance, or version field indicating which correction policies were applied.

In some embodiments, the collection of effective ladder indices is stored as a corrected index vector 516. The corrected index vector 516 may contain one effective value for each sequence position, one value for each codon, one value for each grouped processing unit, or another position-resolved representation. In some embodiments, the corrected index vector 516 is passed directly to the rate calculation engine 112 of FIG. 1. In some embodiments, the corrected index vector 516 is also retained as a user-visible or machine-readable output because it may itself be useful in comparative analysis, modification-effect analysis, quality review, debugging, or downstream design workflows.

The FIG. 5 embodiment is intentionally broad so that the disclosed invention is not limited to a bare single-base mapping. In some embodiments, all correction terms are zero, such that the effective ladder index 514 is identical to the core ladder value. In some embodiments, only nearest-neighbor correction is applied. In some embodiments, only modification-dependent correction is applied. In some embodiments, one or more higher-order or annotation-based corrections are applied. In some embodiments, different correction bundles may be selected for different enzyme classes, operating modes, assay types, or quality-control tasks. In some embodiments, a correction policy may be selected automatically based on metadata. In some embodiments, a correction policy may be supplied by a user, selected by a service profile, or fixed by an implementation version.

The FIG. 5 embodiment therefore provides a deterministic correction framework that supports refinement of core residue-based barrier assignments while preserving computational reproducibility and extensibility. This framework is useful not only for nearest-neighbor and local-context improvements, but also for modified-residue analysis, analog-aware computation, comparative perturbation analysis, and future profile-specific embodiments in which sequence-dependent kinetic behavior is influenced by more than the identity of an isolated residue. By expressly supporting zero-correction, single-correction, and multi-correction pathways, the disclosed invention remains broad enough to cover core implementations and more elaborate embodiments within a single coherent specification.

Referring now to FIG. 6, a representative rate, dwell-time, normalization, and pause-metric computation embodiment is shown. In some embodiments, the corrected index vector 516 generated from the effective-index stage is supplied to a computational pathway that combines an enzyme profile store 600, an attempt frequency nu0 602, a temperature input T 604, and a coherence quantum E_coh 606 to generate one or more position-resolved kinetic outputs. In preferred embodiments, the FIG. 6 embodiment computes a per-position forward rate, a per-position dwell time, a normalized pause density, and one or more derived pause metrics suitable for ranking, clustering, comparison, output generation, or downstream analysis. Although particular components are shown separately in FIG. 6, the disclosed computation may be implemented as a single pipeline, a set of cooperating modules, a vectorized routine, a streaming routine, a hardware-accelerated routine, or another deterministic architecture.

In some embodiments, the enzyme profile store 600 contains one or more profile definitions associated with one or more polymerases, processive molecular machines, assay modes, operating states, calibration bundles, or comparative analysis configurations. The enzyme profile store 600 may be indexed by enzyme identity, enzyme class, assay identifier, template type, user selection, metadata field, service profile, or another profile key. In some embodiments, the enzyme profile store 600 stores a scalar attempt frequency for each profile. In some embodiments, the enzyme profile store 600 stores a structured profile object that includes one or more of an attempt frequency, correction-bundle selection, temperature policy, output policy, implementation policy, or annotation policy. In some embodiments, representative profile entries include, by way of example and not limitation, an RNA polymerase profile, a DNA polymerase profile, a reverse transcriptase profile, a ribosome or codon-based profile, and one or more user-defined or service-defined custom profiles.

In some embodiments, an attempt frequency nu0 602 is obtained from the enzyme profile store 600 or another profile selection pathway. The attempt frequency nu0 602 may be a scalar, a vector, a lookup-driven value, a state-conditioned value, a time-conditioned value, or another profile-linked quantity used in the rate calculation. In preferred embodiments, the attempt frequency nu0 602 is positive and is associated with a selected enzyme profile or assay profile. In some embodiments, the attempt frequency nu0 602 is treated as a profile registry value selected once for a given computation. In some embodiments, the attempt frequency nu0 602 may vary across conditions, profile bundles, or comparative runs. In some embodiments, the disclosed normalized pause metric may remain invariant to constant profile-wide scaling of the attempt frequency nu0 602 across positions, as further described below.

In some embodiments, the temperature input T 604 is received as a scalar thermodynamic condition. In other embodiments, the temperature input T 604 may be supplied as a profile-linked parameter, a record-linked parameter, a time-varying parameter, a position-varying parameter, a condition bundle element, or another quantity associated with the computation. In some embodiments, the temperature input T 604 is specified in Kelvin. In some embodiments, a temperature-conversion stage may convert a received condition into an internal Kelvin-based representation before use in the disclosed equations. In some embodiments, the temperature input T 604 may be selected automatically from a metadata record 318, supplied by user input, associated with a particular assay type, or swept across a plurality of values to generate comparative pause outputs.

In preferred embodiments, the coherence quantum E_coh 606 is supplied as a predefined constant, a stored constant bundle entry, a version-controlled constant value, a computed value derived from a disclosed theoretical chain, or another deterministic barrier quantity. In preferred embodiments, E_coh 606 serves as the quantized coherence-scale barrier unit that links the effective ladder index to the rate calculation. In some embodiments, E_coh 606 is stored once and reused across computations. In some embodiments, E_coh 606 may be generated at runtime, preprocessing time, initialization time, or compile time. In some embodiments, E_coh 606 is provided together with Boltzmann's constant k_B as part of a constant bundle used throughout the computation. In some embodiments, one or more alternate disclosed coherence parameters may be substituted in a manner consistent with the scope of the invention, provided that the resulting computation remains deterministic and traceable.

In some embodiments, the rate calculator 608 receives the effective ladder index 514 or corrected index vector 516 together with the attempt frequency nu0 602, the temperature input T 604, and the coherence quantum E_coh 606, and computes a rate vector k(i) 610. In representative embodiments, the rate calculation is expressed as: k(i; p, T)=nu0(p)*exp(−(n_eff(i)*E_coh)/(k_B*T)) where p identifies the selected profile, n_eff(i) is the effective ladder index at position i, and k_B is Boltzmann's constant. In some embodiments, the rate calculator 608 computes each entry of the rate vector k(i) 610 directly in floating-point form. In some embodiments, the rate calculator 608 computes rates in transformed form, such as log-space form, and later converts them to direct form. In some embodiments, the rate calculation may also be expressed as: log(k(i; p, T))=log(nu0(p))−(n_eff(i)*E_coh)/(k_B*T) which may be useful for numerical stability, vectorization, verification, or comparative analysis. In some embodiments, the rate calculator 608 applies floor rules, ceiling rules, clipping rules, exponent-range rules, precision rules, or other deterministic numerical policies to maintain stable computation across a wide range of sequence lengths and operating conditions.

In preferred embodiments, the rate vector k(i) 610 contains a positive rate for each processed position or unit. In some embodiments, larger effective ladder values correspond to slower rates. In representative embodiments, the relative behavior of two positions may be expressed as: k(i)/k(j)=exp(−((n_eff(i)−n_eff(j))*E_coh)/(k_B*T)) which shows that the ratio of rates depends on the difference in effective ladder index between positions i and j. In some embodiments, this relationship may be used in verification, ranking, quality control, comparative analysis, or profile interpretation. In some embodiments, the rate vector k(i) 610 is exposed as a direct output. In other embodiments, the rate vector k(i) 610 is used internally and not separately reported.

In some embodiments, a dwell-time integrator 612 receives the rate vector k(i) 610 and computes a dwell vector tau(i) 614. In representative embodiments, the dwell-time computation is expressed as: tau(i; p, T)=1/k(i; p, T).

In some embodiments, the dwell vector tau(i) 614 represents a residence time, delay quantity, pause-associated dwell, or another inverse-rate measure for each position. In some embodiments, larger effective ladder values correspond to larger dwell values. In some embodiments, the dwell-time relation between adjacent quantized ladder levels may be expressed as: tau_{n+1}/tau_n=exp(E_coh/(k_B*T)) which indicates exponential spacing between adjacent ladder levels at a given temperature. In some embodiments, the dwell vector tau(i) 614 is retained as an output because it may be useful independently of normalized pause density, such as in timing analysis, folding-window analysis, process simulation, or comparative perturbation analysis.

In some embodiments, a normalizer 616 receives the dwell vector tau(i) 614 and computes a pause-density vector sigma(i) 618. In representative embodiments, the normalized pause metric is expressed as: sigma(i; p, T)=tau(i; p, T)/sum_{j=1 to L} tau(j; p, T) where L is the sequence length or the number of analyzed units. In some embodiments, the pause-density vector sigma(i) 618 is non-negative and sums to one across the analyzed domain, thereby providing a normalized position-resolved pause distribution. In some embodiments, sigma(i) 618 is interpreted as the fraction of aggregate dwell burden or aggregate pause burden associated with position i. In some embodiments, sigma(i) 618 is the preferred pause-pattern output because it preserves position-dependent weighting while allowing clean comparison across computations that differ only by constant scaling of nu0 602.

In some embodiments, where the attempt frequency nu0 602 is constant across positions for a selected profile, the normalized pause metric may be expressed in a form in which nu0 cancels. In a representative embodiment, the normalized relation may be written as: sigma(i; p, T) =exp((n_eff(i)*E_coh)/(k_B*T))/sum_{j=1 to L} exp((n_eff(j)*E_coh)/(k_B*T)).

This expression may be useful in demonstrating that the normalized pause pattern depends on the effective ladder-index structure and temperature, rather than on a constant global scaling of the attempt frequency nu0 602. In some embodiments, this invariance supports cross-profile comparison, transfer analysis, comparative validation, and claims directed to normalized pause behavior. In some embodiments, where nu0 varies by position, state, or another dimension, the general non-cancelled form may instead be used.

In some embodiments, the FIG. 6 embodiment further includes a pause-metric calculator 620 configured to generate one or more derived metrics from the rate vector k(i) 610, the dwell vector tau(i) 614, the pause-density vector sigma(i) 618, or combinations thereof. In some embodiments, the pause-metric calculator 620 computes one or more raw rates, raw dwell values, normalized pause values, windowed scores, cluster scores, differential scores, ratio scores, cumulative burden values, regional burden values, annotation-linked scores, or other pause-related outputs. In representative embodiments, such derived outputs may include: S(i, w)=sum_{u=i−w}\hat{ }{i+w} omega(u−i)*sigma(u) C(W)=sum_{u in W} sigma(u) delta_sigma(i)=sigma_cond2(i)−sigma_cond1(i) R_sigma(i)=sigma_cond2(i)/sigma_cond1(i) where S(i, w) is a windowed local score, C(W) is a cluster or region score over a set W, delta_sigma(i) is a differential pause metric between two conditions, and R_sigma(i) is a ratio-based comparative metric. In some embodiments, omega(u−i) is a weighting kernel, which may be uniform, distance-dependent, triangular, Gaussian-like, policy-driven, or otherwise selected. In some embodiments, the pause-metric calculator 620 computes multiple derived metrics simultaneously and outputs them together or selectively according to a reporting policy.

In some embodiments, the normalized and derived output 622 may therefore include one or more of the rate vector k(i) 610, the dwell vector tau(i) 614, the pause-density vector sigma(i) 618, a ranked site list, a grouped-region score list, a comparative differential map, a ratio map, a windowed score track, a summary report, a browser-compatible output, a machine-readable payload, or another downstream-consumable representation. In some embodiments, the normalized and derived output 622 is transmitted directly to the pause-site calling engine 118 of FIG. 1 or to the output and report module 120. In some embodiments, the normalized and derived output 622 is stored in the data store 124, returned through the API and service interface 122, or passed to a later ranking, clustering, annotation, visualization, or export stage.

The FIG. 6 embodiment may be implemented in a variety of deterministic computational modes. In some embodiments, the full sequence is processed in a batch mode after the entire corrected index vector 516 is available. In some embodiments, the computation is performed incrementally on streamed prefixes or rolling windows. In some embodiments, partial normalization may be performed during streaming, such as by computing: sigma_partial(i, m)=tau(i)/sum_{j=1 to m} tau(j), for i<=m where m represents a current prefix length. In some embodiments, the computation is vectorized over positions, sequences, profiles, temperatures, or condition bundles. In some embodiments, the computation is accelerated using CPUs, GPUs, distributed workers, or specialized numerical hardware. In some embodiments, the numerical implementation uses IEEE-compliant floating-point arithmetic, fixed-point arithmetic, mixed precision, arbitrary precision, log-domain arithmetic, or another explicit deterministic arithmetic policy.

The FIG. 6 embodiment therefore provides the principal kinetic-computation pathway by which an effective ladder representation is converted into one or more usable pause-related outputs. By expressly supporting rate calculation, dwell-time generation, normalization, profile-linked behavior, normalized invariance, differential comparison, and derived-metric computation, the disclosed invention provides robust written-description support for core method claims, normalized-metric claims, comparative claims, profile-based claims, and broader output-family claims within a single coherent computational framework.

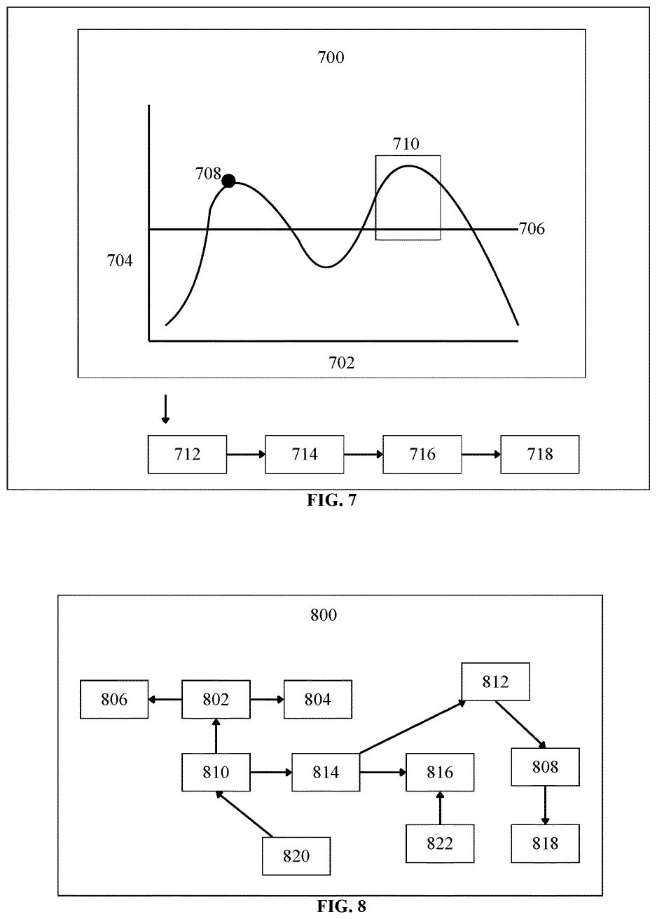

Referring now to FIG. 7, a representative pause-site calling, ranking, clustering, and output-generation embodiment is shown. In some embodiments, the disclosed system receives one or more pause-related inputs, including a pause-density plot 700 or an underlying pause-density vector sigma(i) 618, and processes such inputs to identify one or more positions, intervals, clusters, or regions of interest. In preferred embodiments, the FIG. 7 embodiment operates on one or more outputs from the rate, dwell-time, normalization, and pause-metric computation stage described with reference to FIG. 6 and transforms those outputs into ranked, thresholded, clustered, annotated, or exported results suitable for interpretation, reporting, downstream analysis, or further computational use. Although FIG. 7 illustrates a representative visualization-oriented arrangement, the disclosed functionality may be carried out with or without generating a visible plot and may instead operate directly on stored or streamed numeric values.

In some embodiments, the pause-density plot 700 represents a visual or internal representation of one or more pause-related metrics across sequence position. The pause-density plot 700 may correspond to a normalized pause-density vector, a raw dwell-time vector, a rate-derived inverse score, a local window score, a cluster score, a comparative differential score, or another position-resolved metric. In some embodiments, the pause-density plot 700 is associated with a sequence-position axis 702 and a pause-metric axis 704. The sequence-position axis 702 may correspond to internal sequence indices, genomic coordinates, transcript coordinates, codon positions, feature-relative coordinates, reference offsets, or another positional scheme. The pause-metric axis 704 may correspond to sigma(i), tau(i), a transformed score, a ranked score, a percentile measure, a differential value, or another pause-related quantity.

In some embodiments, pause-site calling is performed using a threshold line 706. The threshold line 706 may correspond to a fixed threshold, a relative threshold, a percentile threshold, an adaptive threshold, a profile-dependent threshold, a sequence-length-dependent threshold, a user-defined threshold, or another decision boundary. In one representative embodiment, a threshold may be expressed as: sigma_threshold=phi/L where phi is the golden ratio and L is the analyzed sequence length. In some embodiments, a position is designated as a pause site when its pause-related value exceeds sigma_threshold. In other embodiments, the threshold line 706 is determined by a percentile of the computed values, by a multiple of a central tendency measure, by a z-score-like normalization, by a windowed score criterion, by a cluster-burden criterion, by a top-N selection rule, or by another explicit deterministic policy. In some embodiments, more than one threshold line 706 may be used, such as a primary threshold and a secondary threshold for candidate refinement, cluster formation, or reporting priority.

In some embodiments, a high-pause position 708 is identified when a single position satisfies one or more calling criteria. For example, a high-pause position 708 may correspond to a local maximum above a threshold line 706, a top-ranked position, a position exceeding a percentile cutoff, or a position identified by a feature-aware rule. In some embodiments, a high-pause position 708 may be output directly as an individual site of interest. In some embodiments, the system identifies a plurality of high-pause positions 708 and ranks them according to one or more metrics, including raw pause density, dwell value, differential score, comparative ratio, local prominence, annotation overlap, or cluster membership.

In some embodiments, a high-pause cluster 710 is identified when multiple nearby positions satisfy one or more cluster criteria. A high-pause cluster 710 may correspond to consecutive above-threshold positions, a merged set of near-adjacent local maxima, a window with elevated aggregate burden, a motif-defined region, a codon group, a regulatory element, or another grouped region of interest. In some embodiments, clustering permits the system to identify broader pause regions rather than isolated point calls. In some embodiments, such clustered regions may better represent biologically meaningful pause zones, pause islands, pause-associated motifs, or design-sensitive regions.

In some embodiments, a cluster merge engine 712 is used to combine, expand, contract, or otherwise process neighboring candidate positions or regions. The cluster merge engine 712 may merge positions separated by less than a specified distance, merge positions falling within the same window, merge positions belonging to the same feature or annotation, merge codon-level signals into a larger region, or otherwise aggregate candidate sites according to a deterministic policy. In some embodiments, the cluster merge engine 712 computes region length, region burden, region score, region rank, centroid position, local maximum position, or feature overlap. In some embodiments, the cluster merge engine 712 may also split clusters where internal spacing, annotation boundaries, or score valleys indicate multiple underlying regions rather than a single merged cluster.

In some embodiments, a ranked site list 714 is generated. The ranked site list 714 may include individual positions, clustered regions, merged regions, or mixed site types ordered according to one or more ranking rules. In some embodiments, ranking is based on sigma(i). In some embodiments, ranking is based on tau(i), S(i, w), C(W), delta_sigma(i), R_sigma(i), a combined score, a weighted score, or another disclosed pause metric. In some embodiments, the ranked site list 714 includes a top-N subset. In some embodiments, the ranked site list 714 includes all called positions above a threshold line 706. In some embodiments, the ranked site list 714 includes both raw values and normalized values, together with coordinate, context, and annotation information.

In some embodiments, an annotation layer 716 is used to associate one or more pause-related outputs with one or more external or internal annotations. The annotation layer 716 may include genomic coordinates, transcript coordinates, codon coordinates, exon or intron labels, promoter or terminator annotations, guide-seed annotations, coding-region annotations, modification annotations, motif annotations, regulatory-region labels, quality-control labels, design-rule labels, or another metadata-linked feature set. In some embodiments, the annotation layer 716 is used only after pause sites have been identified. In other embodiments, the annotation layer 716 may influence the call or ranking itself, such as by prioritizing positions within a selected feature class or by grouping calls by annotation category. In some embodiments, the annotation layer 716 also supports downstream reporting, browser-track generation, comparative genomics output, design screening, and experiment planning.

In some embodiments, an export module 718 is used to generate one or more outputs in a machine-readable, human-readable, or visual format. The export module 718 may output a ranked table, a site list, a cluster list, a coordinate file, a browser-compatible track, a JSON payload, a CSV file, a TSV file, a binary payload, a matrix, a graphical report, an API response, a database record, or another suitable output form. In some embodiments, the export module 718 outputs one or more of the ranked site list 714, an annotation-enriched site list, a clustered-region list, a comparative differential report, a pause-density visualization, or a quality-control summary. In some embodiments, the export module 718 supports local execution, command-line execution, notebook output, API-based delivery, cloud reporting, workflow-engine integration, or storage in an indexed atlas.

In some embodiments, the FIG. 7 embodiment supports multiple site-calling strategies within the same system. A first strategy may use threshold-based calling on sigma(i). A second strategy may use local maxima above the threshold line 706. A third strategy may use a windowed score S(i, w) computed as: S(i, w)=sum_{u=i−w}\hat{ }{i+w} omega(u−i)*sigma(u). A fourth strategy may use a cluster or region score C(W) computed as: C(W)=sum_{u in W} sigma(u). A fifth strategy may use a comparative or perturbation-derived score such as: delta_sigma(i)=sigma_cond2(i)−sigma_cond1(i) or: R_sigma(i)=sigma_cond2(i)/sigma_cond1(i).

In some embodiments, the selected site-calling method is fixed for a deployment. In some embodiments, the selected site-calling method is chosen by a profile, by a use-case mode, by metadata, by a caller, or by another deterministic selection rule. In some embodiments, a plurality of site-calling methods may be applied to the same input to produce parallel outputs. In some embodiments, the FIG. 7 embodiment also supports region aggregation, burden analysis, and feature-aware reporting. For example, the system may aggregate pause density over a promoter region, a terminator region, a guide-seed region, a coding region, a codon window, a transcript segment, a synthetic construct segment, or another designated interval. In some embodiments, such region aggregation is useful for design screening, comparative genomics, therapeutic RNA review, codon optimization, guide ranking, or validation against experimental traces. In some embodiments, the output of the FIG. 7 embodiment is not merely a list of high-scoring points, but a structured analytical product containing site identity, regional context, annotation context, and one or more selected pause-related metrics.

The FIG. 7 embodiment may be used in a wide range of applications. In some embodiments, identified sites or regions correspond to candidate transcriptional pause sites, pause-associated motifs, transcriptional regulatory regions, terminator-associated regions, co-transcriptional folding windows, codon-stall regions, reverse transcriptase stall positions, modified-residue-sensitive sites, or quality-control flags. In some embodiments, the disclosed site-calling and output-generation framework is used in synthetic biology, therapeutic sequence design, vaccine sequence review, transcript engineering, guide RNA screening, comparative genomics, validation of assay conditions, instrument monitoring, or experimental planning. In some embodiments, the same computational framework can be applied to baseline, modified, perturbed, multi-profile, or multi-temperature outputs, thereby allowing direct identification of positions or regions whose pause behavior changes across conditions.

The FIG. 7 embodiment therefore provides a flexible but deterministic post-computation framework for transforming position-resolved pause metrics into actionable site-level and region-level outputs. By supporting thresholding, ranking, clustering, annotation linkage, export, and comparative analysis within a single coherent architecture, the disclosed invention provides strong written-description support for pause-site identification claims, ranked-output claims, clustered-region claims, annotation-linked reporting claims, comparative-output claims, and broader downstream-use embodiments.

Referring now to FIG. 8, a representative computing, API, storage, and non-transitory computer-readable medium embodiment is shown. In some embodiments, the disclosed invention is implemented using a computing system 800 configured to execute one or more deterministic pause-mapping operations on one or more processors and to receive, process, store, transmit, and output one or more sequence-derived pause-related results. The FIG. 8 embodiment is intended to illustrate representative implementation structure for local software, server-side services, cloud deployments, workflow nodes, instrument-connected systems, and machine-readable-medium embodiments. Although particular hardware and software components are shown, the disclosed invention is not limited to any specific physical arrangement, virtualization boundary, operating system, network topology, or deployment architecture.

In some embodiments, the computing system 800 includes a processor 802. The processor 802 may comprise one or more central processing units, graphics processing units, tensor-processing units, vector-processing units, application-specific integrated circuits, field-programmable gate arrays, microcontrollers, system-on-chip devices, or other programmable processing resources. In some embodiments, the processor 802 executes instructions for carrying out one or more of the parser module 106, ladder-index assignment engine 108, effective-index correction engine 110, rate calculation engine 112, dwell-time engine 114, normalization engine 116, pause-site calling engine 118, and output and report module 120 described previously with reference to FIG. 1. In some embodiments, the processor 802 executes batch operations over full sequences or sequence sets. In some embodiments, the processor 802 executes rolling or incremental operations over streamed sequence content. In some embodiments, a plurality of processors 802 may cooperatively execute different portions of the computation.

In some embodiments, the computing system 800 includes a memory 804. The memory 804 may comprise volatile memory, non-volatile memory, random-access memory, cache memory, high-bandwidth memory, unified memory, persistent memory, or combinations thereof. In some embodiments, the memory 804 stores executable code, intermediate data structures, validated sequence arrays 316, metadata records 318, ladder-index arrays 412, corrected index vectors 516, rate vectors k(i) 610, dwell vectors tau(i) 614, pause-density vectors sigma(i) 618, ranked site lists 714, and one or more derived reports or output records. In some embodiments, the memory 804 also stores configuration bundles, constant bundles, profile registries, correction tables, annotation maps, ambiguity policies, and numerical policies used in deterministic execution.

In some embodiments, the computing system 800 includes a persistent storage 806. The persistent storage 806 may comprise a local disk, solid-state storage, network-attached storage, object storage, database-backed storage, sharded storage, distributed storage, or another machine-readable persistence mechanism. In some embodiments, the persistent storage 806 stores input sequence records, computed outputs, profile bundles, correction tables, version manifests, verification artifacts, audit records, benchmark records, compressed pause atlases, browser-compatible tracks, export files, and historical comparison sets. In some embodiments, the persistent storage 806 is used for precomputed atlas embodiments in which pause-related outputs are generated for long templates, transcripts, genomes, libraries, or reference sets and later served by coordinate-based or record-based query.

In some embodiments, the computing system 800 includes a network interface 808. The network interface 808 may comprise one or more wired interfaces, wireless interfaces, cellular interfaces, optical interfaces, inter-process communication interfaces, message-bus interfaces, service-mesh interfaces, or other communication mechanisms that permit exchange of data with remote systems. In some embodiments, the network interface 808 supports communication with client devices, instrument platforms, workflow engines, cloud services, databases, or distributed worker systems. In some embodiments, the network interface 808 is used to receive one or more sequence records, transmit one or more computed outputs, distribute one or more compute jobs, access one or more reference resources, or deliver one or more reports or machine-readable responses.

In some embodiments, the computing system 800 includes an instruction set 810. The instruction set 810 may comprise one or more software modules, executable binaries, scripts, libraries, services, containers, package distributions, firmware images, workflow definitions, or other machine-executable representations of the disclosed methods. In some embodiments, the instruction set 810 includes code that causes the processor 802 to receive a nucleic-acid sequence, parse the sequence, assign ladder indices, compute effective ladder indices, compute rates and dwell times, generate normalized pause metrics, call pause sites or regions, and output one or more deterministic results. In some embodiments, the instruction set 810 is versioned, hashed, signed, or otherwise controlled to support reproducibility, auditing, regression testing, and deployment consistency.

In some embodiments, the computing system 800 includes an application programming interface gateway 812. The application programming interface gateway 812 may expose one or more remote or local interfaces for invoking the disclosed pause-mapping functionality. In some embodiments, the application programming interface gateway 812 supports REST, gRPC, command-line invocation, library invocation, notebook invocation, message-based invocation, workflow-engine invocation, embedded device invocation, or another request/response or streaming interface. In some embodiments, the application programming interface gateway 812 receives requests containing one or more sequence records, one or more temperatures, one or more profile selections, one or more annotation bundles, one or more output options, or one or more batch or streaming instructions. In some embodiments, the application programming interface gateway 812 returns one or more outputs including rate values, dwell values, normalized pause metrics, ranked sites, clustered regions, comparative outputs, reports, browser-compatible tracks, or machine-readable payloads. Endpoint syntax, parameter naming, wire format, and transport format are illustrative and non-limiting.

In some embodiments, the computing system 800 includes a compute worker 814. The compute worker 814 may execute one or more jobs dispatched from the application programming interface gateway 812, a scheduler, a queue, a workflow system, or another orchestration component. In some embodiments, the compute worker 814 processes a single request at a time. In some embodiments, the compute worker 814 processes batches of sequences, batches of temperatures, batches of profile selections, or distributed genome-scale or atlas-scale workloads. In some embodiments, a plurality of compute workers 814 may operate in parallel, such as across multiple processes, containers, nodes, shards, or accelerator-backed workers. In some embodiments, the compute worker 814 is responsible for deterministic execution under a defined constants bundle and algorithm version, even where the wider system is distributed.

In some embodiments, the computing system 800 includes a result store 816. The result store 816 may hold one or more final outputs, partial outputs, cached outputs, historical outputs, comparative outputs, precomputed atlas entries, audit artifacts, benchmark data, or user-accessible reports. In some embodiments, the result store 816 is a database, object store, file set, key-value store, compressed atlas, browser track index, or another storage abstraction. In some embodiments, the result store 816 supports coordinate-based query, record-based query, profile-based query, condition-based query, or version-based query. In some embodiments, the result store 816 is used to serve repeated or large-scale pause queries efficiently without recomputing the entire sequence each time.

In some embodiments, the computing system 800 includes a client interface 818. The client interface 818 may comprise a graphical user interface, command-line interface, programmatic client library, workflow node, notebook widget, browser-accessible interface, instrument dashboard, report viewer, or another mechanism through which a user or upstream system interacts with the disclosed functionality. In some embodiments, the client interface 818 permits input of sequence content, temperatures, enzyme-profile selections, threshold settings, output preferences, modification annotations, comparison requests, or atlas queries. In some embodiments, the client interface 818 presents one or more pause-density plots, ranked site lists, clustered region outputs, comparative maps, design warnings, quality-control reports, or downloadable files. In some embodiments, the client interface 818 is remote from the processor 802. In other embodiments, the client interface 818 is local to the same device or system.

In some embodiments, the computing system 800 includes a non-transitory computer-readable medium 820. The non-transitory computer-readable medium 820 may comprise one or more magnetic, optical, semiconductor, solid-state, flash-based, persistent-memory, or other non-transitory storage media storing machine-readable instructions that, when executed by one or more processors, cause performance of the disclosed methods. In some embodiments, the non-transitory computer-readable medium 820 stores the instruction set 810, one or more profile bundles, one or more correction bundles, one or more constants bundles, one or more verification artifacts, or one or more deployment manifests. In some embodiments, the non-transitory computer-readable medium 820 forms the basis of a computer-readable-medium claim directed to instructions for receiving a sequence, assigning ladder values, computing rate and dwell outputs, determining normalized pause metrics, identifying sites or regions, and outputting one or more results.

In some embodiments, the computing system 800 includes a checksum and version engine 822. The checksum and version engine 822 may compute one or more hashes, checksums, digital signatures, manifests, profile identifiers, constant-bundle identifiers, correction-policy identifiers, algorithm-version identifiers, or other provenance indicators associated with a computation or its outputs. In some embodiments, the checksum and version engine 822 supports bit-identical reproducibility testing across deployments. In some embodiments, the checksum and version engine 822 supports numerical-equivalence auditing within a defined tolerance. In some embodiments, the checksum and version engine 822 is used to verify that a given output was generated with a specified instruction set 810, profile definition, numerical policy, and constants bundle. In some embodiments, the checksum and version engine 822 may attach such information to an API response, store it in the result store 816, include it in a report, or provide it for later audit comparison.

The FIG. 8 embodiment supports a variety of implementation modes. In some embodiments, the disclosed invention is implemented as a local software library executing entirely on a single device. In some embodiments, the disclosed invention is implemented as a command-line tool that reads a sequence file and outputs pause-related results. In some embodiments, the disclosed invention is implemented as a network-accessible API service in which the application programming interface gateway 812 receives requests, the compute worker 814 executes deterministic computation, and the result store 816 stores or serves one or more outputs. In some embodiments, the disclosed invention is implemented as a batch-processing system for large sequence collections, a distributed atlas-generation pipeline, or a real-time streaming service coupled to an instrument or base-calling platform. In some embodiments, the same core computational logic is reused across local, remote, embedded, and distributed deployments.

In some embodiments, determinism is supported by explicit implementation policies. Such policies may include use of a defined constants bundle, use of a defined correction-policy bundle, elimination of stochastic sampling, elimination of sequence-specific machine-learning training during runtime execution, elimination of iterative parameter fitting, canonical parsing behavior, explicit arithmetic policy selection, explicit profile selection, and explicit version tracking. In some embodiments, the system is configured to generate bit-identical results for identical inputs under a fixed deployment policy. In other embodiments, the system is configured to generate results that are numerically equivalent within a defined tolerance while still preserving deterministic algorithmic structure. In some embodiments, such determinism-supporting policies are stored in the metadata record 318, the instruction set 810, the result store 816, the checksum and version engine 822, or combinations thereof.

In some embodiments, the computing system 800 may further support security, access control, auditing, job scheduling, queueing, fault tolerance, retry policy, caching, and distribution features without changing the substantive computational framework disclosed herein. In some embodiments, one or more components shown in FIG. 8 may be combined. In some embodiments, one or more components may be omitted. In some embodiments, one or more functions associated with one component may instead be carried out by another component. For example, the application programming interface gateway 812 and client interface 818 may be combined in a single service layer, or the result store 816 and persistent storage 806 may be implemented through a shared storage substrate.

The FIG. 8 embodiment therefore provides representative implementation support for system claims, service claims, local-execution claims, API claims, batch-processing claims, atlas claims, and non-transitory computer-readable-medium claims. By expressly disclosing processors, memory, storage, interfaces, workers, versioning, provenance, and machine-readable instructions within a unified architecture, the specification supports a wide range of practical commercial and technical implementations while preserving a coherent relationship to the sequence-processing, ladder-mapping, effective-index, kinetic-computation, and site-calling embodiments described with reference to FIGS. 1 through 7.

In some embodiments, the disclosed invention further includes a formal verification embodiment in which one or more properties of the computational framework are checked, proved, certified, or otherwise validated using a formal reasoning system, a symbolic reasoning environment, a model checker, a theorem prover, a proof assistant, a rule engine, or another verification framework. The formal verification embodiment may be used to confirm that one or more aspects of the disclosed sequence-processing, ladder-mapping, effective-index, rate-calculation, dwell-time, normalization, site-calling, or implementation logic satisfy predetermined correctness conditions. In preferred embodiments, the formal verification embodiment is optional and supports confidence, auditability, reproducibility, and prosecution robustness, but does not limit the invention to any particular proof environment or certification requirement.

In some embodiments, the formal verification embodiment is configured to verify one or more ladder-index properties. Such properties may include boundedness of the core ladder mapping, boundedness of a selected corrected-index policy, validity of canonical residue assignments, correctness of ambiguity handling, consistency of deterministic parsing rules, consistency of strand-selection policies, consistency of coordinate-preservation rules, or determinism of the preprocessing pipeline. In some embodiments, a verification environment may be used to confirm that the core residue-to-index mapping preserves one or more expected relationships, such as the assignment of adenine, thymine, uracil, guanine, and cytosine to distinct ladder values under a selected mapping policy. In some embodiments, one or more correction policies may also be verified to ensure that corrected values remain within a selected permitted domain or satisfy one or more monotonicity, quantization, or provenance rules.

In some embodiments, the formal verification embodiment is configured to verify one or more rate properties associated with the disclosed rate calculation. Such properties may include positivity of the per-position rate, monotonic dependence of rate on effective ladder index under fixed temperature and constant bundle, valid dependence on a positive attempt-frequency term, or consistency of transformed and non-transformed arithmetic forms. In some embodiments, the verification environment may be used to confirm that, for positive nu0 and positive T, the expression: k(i; p, T)=nu0(p)*exp(−(n_eff(i)*E_coh)/(k_B*T)) produces a positive value for each analyzed position. In some embodiments, the formal verification embodiment may confirm that when n_eff(i) exceeds n_eff(j) under otherwise fixed conditions, the resulting rate at position i is lower than the rate at position j. In some embodiments, such formalized results may support implementation testing, deployment validation, regression control, or explanatory documentation.

In some embodiments, the formal verification embodiment is configured to verify one or more dwell-time properties. Such properties may include positivity of dwell values, inverse consistency between rate and dwell expressions, monotonic increase of dwell with increasing effective ladder index, exponential ratio behavior between adjacent ladder levels under fixed temperature, or consistency of direct-form and transformed-form dwell calculations. In some embodiments, the verification environment may be used to confirm that: tau(i; p, T)=1/k(i; p, T) is positive whenever the corresponding rate is positive. In some embodiments, the verification environment may also be used to confirm that a higher effective barrier yields a longer dwell under otherwise fixed conditions.

In some embodiments, the formal verification embodiment is configured to verify one or more normalization properties associated with the normalized pause metric. Such properties may include non-negativity of sigma(i), normalization of sigma(i) over the analyzed domain, correct cancellation of a constant profile-wide attempt frequency in a normalized formulation, equivalence between direct normalization and transformed normalization, or preservation of one or more ordering relations under normalization. In some embodiments, the verification environment may be used to confirm that: sigma(i; p, T)=tau(i; p, T)/sum_{j=1 to L} tau(j; p, T) is non-negative and sums to one over the relevant positions, provided that the denominator is positive. In some embodiments, the formal verification embodiment may further verify that where nu0 is constant across positions, the normalized pattern depends on effective ladder structure and temperature rather than on the constant scaling factor itself.

In some embodiments, the formal verification embodiment is configured to verify one or more determinism properties. Such properties may include functional determinism for a fixed input, constants bundle, profile bundle, correction bundle, numerical policy, and algorithm version; consistency between repeated runs; consistency across local and distributed execution under a specified deterministic arithmetic policy; consistency of checksum generation; consistency of version tracking; or consistency of selected export transformations. In some embodiments, the verification environment may be used to prove or check that identical accepted inputs under a fixed execution policy yield identical outputs. In some embodiments, this may include verification of bit-identical output under a defined arithmetic environment. In other embodiments, this may include verification of numerical equivalence within a specified tolerance bound.

In some embodiments, the formal verification embodiment is configured to verify one or more site-calling or output-generation properties. Such properties may include deterministic threshold application, deterministic cluster formation, deterministic ranking rules, deterministic export formatting, monotonic ranking under selected score transformations, or consistency of coordinate mapping between internal indices and output records. In some embodiments, the verification environment may be used to confirm that a selected thresholding rule, percentile rule, or clustering rule yields a stable and reproducible site list for a given input and parameter set. In some embodiments, the formal verification embodiment may also be used to confirm that output records preserve expected provenance fields, profile identifiers, coordinate mappings, and version labels.

In some embodiments, the formal verification embodiment is implemented using Lean. In some embodiments, a Lean-based formalization may be used to prove one or more propositions regarding ladder-index boundedness, positivity of rates, positivity of dwell times, monotonic ordering, normalization, determinism, or invariance. In other embodiments, another theorem prover, proof assistant, symbolic engine, model checker, satisfiability engine, algebraic verification environment, proof-carrying-code environment, or custom verification framework may be used. The invention is not limited to Lean or to any particular formal language, syntax, proof style, certificate structure, or mathematical library.

In some embodiments, the formal verification embodiment produces one or more artifacts, including a proof script, a proof object, a compiled certificate, a theorem list, a verification manifest, a hash-linked certification record, a deployment attestation, a report, or another machine-readable or human-readable verification output. In some embodiments, such artifacts may be stored in the persistent storage 806, attached to a result store 816, versioned through the checksum and version engine 822, distributed with the instruction set 810, or otherwise associated with a particular software release or constants bundle. In some embodiments, a verification artifact may be used to demonstrate that a deployed implementation conforms to one or more formally checked mathematical or logical properties.

In some embodiments, formal verification is applied only to selected parts of the disclosed system, such as the core mapping and normalization logic. In some embodiments, formal verification is applied to a broader set of modules, including parsing, context correction, modified-residue handling, output generation, or service behavior. In some embodiments, formal verification is used during development and test but not distributed with a production deployment. In some embodiments, verification artifacts are distributed together with a deployed service or library. In some embodiments, verification may be repeated after a profile update, correction-bundle update, numerical policy update, or algorithm-version change.

The disclosed formal verification embodiment provides additional support for implementations in regulated, commercial, scientific, or high-confidence environments by enabling explicit mathematical or logical checking of selected properties of the pause-mapping framework. By disclosing optional verification of ladder mapping, rate computation, dwell computation, normalization, determinism, output consistency, and related properties, the specification supports embodiments in which correctness assurance forms part of the implementation or release workflow without requiring that every embodiment of the invention include a formal proof subsystem.

The following examples are provided to illustrate representative embodiments of the disclosed invention and are not intended to limit the scope of the invention. Unless otherwise indicated, an example may represent an illustrative calculation, a simulated implementation, a prophetic implementation, an experimentally informed implementation, or a combination thereof. A person of ordinary skill in the art will appreciate that the disclosed methods can be applied to other sequences, temperatures, profiles, correction bundles, annotation inputs, and implementation environments while remaining within the scope of the invention.

In a first illustrative example, the disclosed method is applied to a short RNA-polymerase sequence-processing case using a sequence having ten positions. In one representative implementation, the sequence is: ATGCAATGCC.

The sequence may be received through the sequence input source 102, parsed by the parser module 106, canonicalized and validated according to the preprocessing pathway described with reference to FIG. 3, and converted into a core ladder-index representation according to the residue-to-ladder mapping described with reference to FIG. 4. Under the preferred core mapping, the corresponding ladder assignments are:
n(A)=0
n(T)=1
n(G)=2
n(C)=3
   so that the resulting core ladder-index sequence is: [0, 1, 2, 3, 0, 0, 1, 2, 3, 3].

In an illustrative implementation in which no correction term is applied, the effective ladder indices are equal to the core ladder indices. For a selected profile p corresponding to T7 RNA polymerase, with nu0(p)=260 s\hat{ }−1 and T=298 K, the rate calculation engine 112 may compute the per-position forward rates according to: k(i; p, T)=nu0(p)*exp(−(n_eff(i)*E_coh)/(k_B*T)).

Using E_coh approximately equal to 0.090 eV and k_B approximately equal to 8.617×10\hat{ }−5 eV/K, representative position-wise rate values may be calculated as approximately 260.0 s\hat{ }−1 for positions assigned A, approximately 7.87 s\hat{ }−1 for positions assigned T, approximately 0.238 s\hat{ }−1 for positions assigned G, and approximately 7.20×10\hat{ }−3 s\hat{ }−1 for positions assigned C. The dwell-time engine 114 may then compute dwell values according to: tau(i; p, T)=1/k(i; p, T) yielding representative dwell values of approximately 0.00385 seconds for A positions, approximately 0.127 seconds for T positions, approximately 4.20 seconds for G positions, and approximately 138.9 seconds for C positions. The normalization engine 116 may then compute a normalized pause-density output according to: sigma(i; p, T)=tau(i; p, T)/sum_{j=1 to L} tau(j; p, T) where L=10 for this sequence. In this representative example, the resulting normalized pause distribution is dominated by the cytosine positions and demonstrates that the disclosed method can identify major pause-contributing sites from sequence content alone. This first example is an illustrative calculation.

In a second illustrative example, the disclosed method is used to evaluate temperature dependence. A short sequence may be processed at multiple temperatures while holding sequence content and selected profile constant. For example, a sequence such as: ATGC may be evaluated for a selected profile p with nu0(p)=260 s\hat{ }−1 at T=298 K, T=310 K, and T=370 K. For a cytosine position, the resulting rate may increase as temperature increases, while the corresponding dwell time decreases. In one representative implementation, a cytosine position may produce a dwell time on the order of approximately 138.9 seconds at 298 K, approximately 65.0 seconds at 310 K, and approximately 2.29 seconds at 370 K, depending on the exact constant values and arithmetic policy used. This example illustrates that the disclosed framework preserves the expected monotonic temperature dependence of rate and dwell. This second example is an illustrative calculation.

In a third example, the disclosed method is applied to a promoter-associated region or other annotated transcriptional region. A sequence record 300 may contain a selected genomic interval, promoter region, transcript segment, or engineered construct segment. The sequence is processed through the parser module 106, the ladder-index assignment engine 108, the effective-index correction engine 110, the rate calculation engine 112, the dwell-time engine 114, the normalization engine 116, and the pause-site calling engine 118. The output and report module 120 may then identify one or more high-pause positions 708 or one or more high-pause clusters 710 that overlap with sequence regions of elevated GC burden, consecutive high-rung residues, codon-sensitive regions, or other pause-associated features. In some embodiments, the ranked site list 714 may be compared against experimentally observed pause positions, annotation features, or known regulatory regions. This third example may be implemented as an experimentally informed or prophetic example, depending on the data source used.

In a fourth example, the disclosed method is applied to a long template, such as a viral genome, plasmid, synthetic construct, transcript panel, or another extended sequence. The validated sequence array 316 may be generated for a template having tens of thousands, hundreds of thousands, or millions of positions. The lookup engine 410 generates a ladder-index array 412, the effective ladder-index correction stage generates a corrected index vector 516, and the FIG. 6 computation stage generates one or more rate, dwell, normalized pause, and derived metric outputs for the full template. The resulting ranked site list 714 may be used to identify candidate transcription terminators, pause-rich regions, or design-sensitive intervals. In some embodiments, the same example may also demonstrate the linear-time sequence-processing properties of the disclosed framework. This fourth example may be implemented as an illustrative large-scale computation, a benchmark example, or a prophetic genome-scale analysis example.

In a fifth example, the disclosed method is used in guide-sequence screening. A plurality of candidate guide sequences, target regions, protospacer-adjacent motif combinations, or guide-associated transcription templates may be received as a batch input through the sequence record 300. Each candidate is processed through the disclosed pause-mapping framework to generate one or more pause-related outputs, including one or more local pause metrics within a seed region or target-sensitive region. In some embodiments, candidates with lower local pause burden within a selected region are ranked more favorably for downstream use. In some embodiments, the disclosed method is used as one factor among several guide-screening criteria. In some embodiments, the output is returned as a ranked candidate table generated by the export module 718. This fifth example is a prophetic or design-oriented example.

In a sixth example, the disclosed method is used for comparative perturbation analysis. A baseline sequence and a modified or perturbed sequence may be processed separately and compared using the pause-metric calculator 620. In some embodiments, the perturbed condition corresponds to a profile change, a temperature change, a modification-aware processing mode, an analog-containing sequence, or a chemical-perturbation model. In some embodiments, one or more comparison outputs are generated according to: delta_sigma(i)=sigma_cond2(i) sigma_cond1(i) and: R_sigma(i)=sigma_cond2(i)/sigma_cond1(i).

In some embodiments, the resulting output identifies positions or regions whose pause behavior changes under the selected perturbation. In some embodiments, a perturbation that changes nu0(p) by a constant factor across all positions changes absolute dwell values while preserving a normalized pause-density pattern. In other embodiments, a perturbation that changes one or more effective ladder-index terms changes the normalized pattern itself. This sixth example is an illustrative comparative example and may also be used as a prophetic drug-screening or condition-screening example.

In a seventh example, the disclosed method is used in a modified-residue or therapeutic-RNA quality-control workflow. A sequence record 300 may include one or more modification annotations parsed by the modified-residue parser 314, and a modification correction may be applied through the modification correction store 510. In some embodiments, the effective ladder index 514 is altered at one or more positions according to: n_eff(i)=n(i)+delta_mod(m_i) and the resulting corrected index vector 516 is used to generate rates, dwell values, normalized pause values, and one or more comparative metrics. In some embodiments, the analysis is used to identify whether a modified residue or residue pattern increases local pause burden, decreases local pause burden, or redistributes pause behavior across a designed transcript. In some embodiments, the method is used to evaluate pseudouridine substitutions, methylated residues, nucleotide analogs, or other modified bases in a therapeutic RNA, vaccine RNA, or engineered construct. This seventh example is a prophetic and design-oriented example, although it may also be implemented with experimental data.

In an eighth example, the disclosed method is used in a real-time streaming context. A stream of base calls, chunks, or partial sequence segments is received as an incremental sequence record 300 and processed through the format detector 304, the canonicalizer 306, and the validated sequence array 316 in a rolling or chunked fashion. The lookup engine 410 and the FIG. 6 computational stages may then compute partial or rolling outputs as the sequence arrives. In some embodiments, a partial normalized output is computed according to: sigma_partial(i, m)=tau(i)/sum_{j=1 to m} tau(j), for i<=m where m is a current prefix length. In some embodiments, the client interface 818 presents a live or near-real-time pause profile, and one or more high-pause positions 708 or high-pause clusters 710 are flagged during sequence acquisition. This eighth example is a prophetic implementation example and supports real-time sequencing and instrument-connected embodiments.

In a ninth example, the disclosed method is adapted to a codon or translation-oriented embodiment. A nucleic-acid sequence may be parsed into codon units, and the disclosed residue-to-index mapping may be aggregated at the codon level. In some embodiments, a codon-level ladder value is expressed as: n_codon(c)=n(b_1)+n(b_2)+n(b_3)+delta_codon(c) where b_1, b_2, and b_3 are the residues of codon c. A codon-level rate may then be computed according to: k_codon(c, T)=nu0_ribo*exp(−(n_codon(c)*E_coh)/(k_B*T)).

In some embodiments, resulting outputs include codon stall scores, codon-level pause densities, local translation-burden scores, or codon optimization recommendations. In some embodiments, synonymous codon variants are compared to identify coding designs associated with more favorable pause patterns in selected regions. This ninth example is a prophetic codon-optimization and translation example.

In a tenth example, the disclosed method is applied to an alternate enzyme class, such as a reverse transcriptase or DNA polymerase. The same overall pipeline is used, but the selected profile p corresponds to a different enzyme class and may include a different attempt frequency nu0(p), a different correction bundle, a different annotation policy, or a different operating mode. In some embodiments, the resulting outputs are used to identify stall-prone sites, polymerase-sensitive regions, sequence features associated with altered copying kinetics, or regions of interest for assay optimization. In some embodiments, this example demonstrates that the disclosed framework is not limited to RNA polymerase and can be extended across multiple processive molecular machines using the same core barrier-mapping architecture. This tenth example is a prophetic alternate-enzyme example.

In some embodiments, one or more of the foregoing examples may be combined. For example, a modified-residue sequence may be evaluated across multiple temperatures and multiple enzyme profiles, or a long-template atlas computation may be coupled to a site-calling and annotation workflow. In some embodiments, examples may be implemented locally on a single computing system 800. In some embodiments, examples may be implemented through the application programming interface gateway 812 and one or more compute workers 814. In some embodiments, examples may be performed in a batch workflow, a notebook environment, a cloud service, or a real-time instrument-connected deployment.

These examples illustrate that the disclosed invention supports a broad family of deterministic sequence-to-pause computations, including short-sequence calculations, temperature-dependent analysis, profile-based analysis, comparative perturbation analysis, modified-residue analysis, real-time streaming, codon-level analysis, and alternate-enzyme embodiments, all within a single coherent computational framework.

In some embodiments, the disclosed invention is further supported by validation and benchmarking workflows configured to evaluate predictive performance, computational performance, reproducibility, transfer behavior across enzyme profiles, and consistency under comparative conditions. Such workflows may be used during development, deployment, quality assurance, scientific validation, regulatory support, commercial evaluation, or post-deployment monitoring. The validation and benchmarking embodiments described herein are illustrative and non-limiting, and a person of ordinary skill in the art will appreciate that other validation protocols, assay platforms, scoring criteria, and benchmark datasets may be used while remaining within the scope of the invention.

In some embodiments, validation is performed by comparing one or more computed outputs of the disclosed system against one or more experimentally observed pause-related measurements. Such measurements may include polymerase dwell traces, optical-tweezer measurements, sequencing-based pause measurements, NET-seq-derived signals, mNET-seq-derived signals, run-on transcription measurements, real-time instrument traces, reverse transcriptase stall traces, ribosome profiling signals, or other position-resolved or region-resolved measurements reflecting sequence-dependent kinetic behavior. In some embodiments, the computed outputs used for comparison include the rate vector k(i) 610, the dwell vector tau(i) 614, the pause-density vector sigma(i) 618, a ranked site list 714, a cluster or region score, a comparative differential map, or another pause-related output generated by the disclosed system.

In some embodiments, a validation workflow uses a reference sequence and a computed pause-related output generated by the disclosed sequence-processing and pause-mapping framework together with an experimental trace aligned to the same template. In some embodiments, the reference sequence corresponds to a genomic region, transcript region, synthetic construct, viral genome, plasmid, guide set, therapeutic sequence, codon-optimized construct, or another experimentally testable template. The computed pause-related output may include one or more of a rate vector, dwell vector, pause-density vector, ranked site list, clustered region output, or derived pause metric, and the experimental trace may comprise one or more observed kinetic or occupancy measurements aligned to the same template. In some embodiments, the validation workflow is implemented using the components described with reference to FIGS. 1 through 8.

In some embodiments, an alignment procedure is used to align computed outputs with experimental observations. The alignment procedure may map the sequence-position axis 702 of a computed output to genomic coordinates, transcript coordinates, codon positions, feature-relative coordinates, or instrument trace positions. In some embodiments, alignment accounts for offsets, clipping, filtering, strand interpretation, template orientation, coordinate conventions, read trimming, or partial-window overlap. In some embodiments, the alignment procedure may be used before comparison of a computed pause-related output with an experimental trace, or before comparison between two computed outputs under different conditions.

In some embodiments, one or more quantitative metrics are used to assess validation performance. Such metrics may include correlation, rank correlation, root-mean-square error, mean absolute error, top-site overlap, cluster overlap, thresholded-call overlap, precision, recall, F-score, ranking consistency, latency, throughput, reproducibility, or another quantitative comparison measure. In some embodiments, a correlation metric may evaluate similarity between a predicted pause-density vector sigma(i) 618 and a position-resolved experimental signal. In some embodiments, a root-mean-square or mean absolute deviation metric may evaluate agreement between predicted dwell values and experimentally measured dwell times. In some embodiments, a top-site overlap metric may evaluate how many of the highest-ranked predicted positions correspond to experimentally identified pause sites or regions. In some embodiments, a cluster-overlap metric may assess agreement between predicted high-pause clusters 710 and experimentally observed regions of elevated pausing or occupancy.

In some embodiments, optical-tweezer validation is performed. A template sequence may be processed by the disclosed system to generate one or more predicted rates, dwell values, and pause metrics, and those computed quantities may then be compared with single-molecule dwell measurements from one or more selected positions along the same template. In some embodiments, the comparison is performed position by position. In some embodiments, the comparison is performed after smoothing, windowing, or clustering. In some embodiments, the disclosed system is considered to satisfy a selected validation criterion when one or more error measures fall below a selected threshold, when one or more rank measures exceed a selected threshold, or when one or more predicted high-pause positions align with observed high-dwell positions according to a defined policy.

In some embodiments, sequencing-based validation is performed. For example, a genome segment, transcript collection, or promoter-associated region may be processed to generate one or more predicted pause-related outputs, and the resulting outputs may be compared to one or more sequencing-based pause or occupancy datasets. In some embodiments, such comparison may use normalized values, ranked values, region-level summaries, or thresholded site calls. In some embodiments, the disclosed system may be evaluated on its ability to identify pause-rich regions, cluster boundaries, regulatory regions, coding-region pauses, or other biologically meaningful pause signatures reflected in sequencing-based assays.

In some embodiments, temperature titration validation is performed. A sequence may be analyzed at multiple temperatures using the disclosed framework, and one or more predicted rate ratios, dwell ratios, normalized patterns, or region-level burdens may be compared against experimentally observed temperature-dependent behavior. In some embodiments, the comparison may be used to assess whether increasing temperature produces the predicted monotonic increase in rate and decrease in dwell. In some embodiments, the comparison may also be used to assess whether relative pause ordering is preserved across a selected temperature range or whether certain regions exhibit increased or decreased pause prominence under altered conditions.

In some embodiments, cross-profile or cross-enzyme validation is performed. For example, the same sequence may be processed under two different profile selections p1 and p2, each with a respective attempt-frequency value or profile bundle. In some embodiments, where the attempt-frequency scaling differs by a constant factor across all positions but the effective ladder structure remains unchanged, the normalized pause metric may be expected to remain invariant or substantially invariant. In some embodiments, this comparative property is validated by showing that sigma(i; p1, T) and sigma(i; p2, T) agree exactly or within a selected tolerance under such conditions. In some embodiments, such cross-profile validation supports the claimed distinction between absolute timing changes and normalized pause-pattern invariance.

In some embodiments, modified-residue validation is performed. A baseline sequence and a modified sequence may be compared using one or more perturbation metrics generated by the pause-metric calculator 620, including delta_sigma(i), delta_tau(i), or related difference measures. In some embodiments, the comparison is used to determine whether a selected modification policy produces predicted local or regional changes in pause burden. In some embodiments, modified-residue validation may be relevant for therapeutic RNA review, vaccine-sequence quality control, analog-containing constructs, or engineered sequence optimization.

In some embodiments, site-calling validation is performed. One or more high-pause positions 708, high-pause clusters 710, ranked site lists 714, or exported outputs from the export module 718 may be compared with experimentally observed pause sites, regulatory motifs, known terminators, observed stall regions, guide-sensitive regions, or another annotated truth set or reference set. In some embodiments, validation includes assessment of threshold sensitivity, ranking stability, cluster merge behavior, annotation overlap, or false-positive and false-negative behavior under a selected call policy. In some embodiments, multiple site-calling policies are benchmarked in parallel to determine which policy is preferable for a given use case.

In some embodiments, computational benchmarking is performed in addition to or instead of biological validation. Computational benchmarking may evaluate runtime, throughput, memory usage, storage usage, streaming latency, atlas-generation time, distributed scaling behavior, request latency, or another implementation metric. In some embodiments, benchmarking is performed on one or more processors 802, one or more compute workers 814, one or more local devices, one or more cloud instances, one or more GPUs, or one or more distributed worker sets. In some embodiments, runtime is measured for one or more templates of different lengths. In some embodiments, throughput is measured as processed positions or residues per unit time. In some embodiments, latency is measured as time to first result, time to complete result, streaming update latency, or API round-trip latency. In some embodiments, memory usage is measured for one or more implementations of the validated sequence array 316, ladder-index array 412, corrected index vector 516, or result store 816.

In some embodiments, atlas benchmarking is performed. In such embodiments, the disclosed system generates pause-related outputs for large reference templates, large sequence libraries, transcriptomes, or genomes and stores the resulting outputs in persistent storage 806 or result store 816. Benchmarking may evaluate total atlas-generation time, shard-level scaling, compression ratio, query latency, coordinate-based retrieval time, cache efficiency, or update cost for profile changes, annotation changes, or correction-bundle changes. In some embodiments, atlas benchmarking supports large-scale commercial or research deployments in which repeated query of precomputed results is preferable to repeated recomputation.

In some embodiments, streaming benchmarking is performed. The disclosed system may receive base calls or sequence chunks incrementally, and performance may be assessed in terms of rolling-update speed, partial-normalization latency, event-to-alert time, or other streaming-specific metrics. In some embodiments, such benchmarking is relevant where the disclosed system is coupled to an instrument, a base-calling pipeline, a monitoring workflow, or an alerting system. In some embodiments, streaming validation and benchmarking may be combined, such as by comparing live computed outputs against a later finalized reference profile.

In some embodiments, reproducibility benchmarking is performed. The same input sequence, profile selection, temperature, constants bundle, correction bundle, and algorithm version may be processed multiple times through the same deployment or across different deployments. In some embodiments, the resulting outputs are compared using hashes, checksums, exact numeric comparison, or tolerance-based comparison. In some embodiments, the checksum and version engine 822 is used to confirm bit-identical or numerically equivalent reproduction of outputs. In some embodiments, reproducibility benchmarking is used to validate library releases, service deployments, accelerator-specific implementations, or numerical-policy changes.

In some embodiments, validation and benchmarking results are collected into one or more reports, dashboards, comparison records, benchmark stores, or audit records. Such outputs may be stored in persistent storage 806, result store 816, or another reporting subsystem. In some embodiments, a validation record includes the tested sequence or sequence set, the selected profile, the selected temperature or temperature range, the selected correction policy, the selected call policy, the selected constants bundle, the selected algorithm version, one or more measured metrics, and one or more timestamps or provenance fields. In some embodiments, such records may be used to support deployment qualification, internal quality review, publication support, customer support, regulatory documentation, or litigation support.

The disclosed validation and benchmarking embodiments therefore provide structured support for evaluating both scientific and computational aspects of the invention. By expressly supporting comparison to experimental traces, temperature titration, profile invariance testing, modified-residue comparison, site-calling evaluation, runtime benchmarking, atlas benchmarking, streaming benchmarking, and reproducibility auditing, the specification supports embodiments in which the disclosed deterministic pause-mapping framework is not only computed, but also measured, tested, verified in practice, and performance-characterized across a wide variety of use cases.

The embodiments described herein are illustrative and non-limiting. Although the invention has been described with reference to particular computational stages, data structures, equations, profiles, correction pathways, output types, deployment models, and example use cases, a person of ordinary skill in the art will appreciate that numerous variations, substitutions, extensions, and rearrangements may be made without departing from the scope of the invention as defined by the claims. The invention is therefore not limited to the specific embodiments expressly described, except as may be required by the claims as finally allowed.

In some embodiments, one or more modules described herein may be combined into a single component, subdivided into multiple components, distributed across multiple devices, virtualized, containerized, parallelized, accelerated, or otherwise implemented in a different architectural arrangement. For example, the parser module 106, ladder-index assignment engine 108, effective-index correction engine 110, rate calculation engine 112, dwell-time engine 114, normalization engine 116, pause-site calling engine 118, and output and report module 120 may be implemented as distinct modules, partially merged modules, a single software pipeline, a service mesh, a workflow graph, a library call chain, a hardware-assisted process, or another suitable implementation. Likewise, the application programming interface gateway 812, compute worker 814, result store 816, and client interface 818 may be implemented in a unified service architecture or in a distributed architecture.

The disclosed computational steps need not be performed in the exact order in which they are described, unless a particular order is expressly required by the logic of a given embodiment or by a claim. In some embodiments, parsing, canonicalization, ambiguity handling, coordinate preservation, and modification handling may be performed in a different sequence. In some embodiments, correction calculations may be applied before, during, or after one or more other processing stages. In some embodiments, rate, dwell, normalization, and derived pause metrics may be computed in direct sequence, partially in parallel, or in transformed form and later converted to a reporting form. In some embodiments, ranking, clustering, annotation association, and export may occur as a single integrated post-processing stage or as multiple separate stages.

The disclosed equations may be implemented in direct arithmetic form, transformed form, normalized form, scaled form, log-space form, approximate form, exact form, vectorized form, lookup-assisted form, mixed-precision form, or another computationally suitable form, provided that the resulting computation corresponds to the disclosed relationships. For example, the disclosed rate equation may be evaluated directly, evaluated in log space and exponentiated later, evaluated through a lookup structure, or evaluated through an equivalent monotonic transform and inverse transform. Likewise, normalized pause metrics may be computed through direct summation, streaming accumulation, blockwise accumulation, parallel reduction, compensated summation, or another deterministic or numerically controlled summation strategy.

The disclosed data representations are likewise illustrative rather than limiting. A sequence, ladder-index array, corrected index vector, rate vector, dwell vector, pause-density vector, ranked site list, metadata record, or annotation record may be stored or represented as an array, vector, list, table, matrix, map, object, tuple, record, stream, chunked buffer, sparse structure, packed representation, graph-linked representation, or another suitable machine-readable format. In some embodiments, one or more such data structures may be transient. In some embodiments, one or more such data structures may be persistent. In some embodiments, one or more such data structures may be compressed, encrypted, sharded, cached, or streamed.

The disclosed system is not limited to any particular programming language, framework, operating system, deployment platform, processor architecture, cloud vendor, network protocol, storage engine, or interface standard. In some embodiments, the invention is implemented in a compiled language. In some embodiments, the invention is implemented in an interpreted language. In some embodiments, the invention is implemented in multiple interoperating languages. In some embodiments, the invention is implemented as a local executable, a shared library, a package, a container, a web service, a serverless function, a notebook-accessible engine, an embedded process, an instrument-connected component, or another software or firmware embodiment. In some embodiments, one or more disclosed computations may be accelerated using vector instructions, graphics processors, tensor processors, field-programmable gate arrays, application-specific integrated circuits, or other specialized hardware.

The disclosed input and output formats are also illustrative and non-limiting. Although examples herein refer to plain text, FASTA, FASTQ, GenBank, structured object payloads, batch payloads, and streamed sequence content, other input representations may also be used. Likewise, although examples herein refer to JSON, CSV, TSV, browser-compatible tracks, reports, tables, and machine-readable payloads, other output representations may also be used. Endpoint syntax, field naming, parameter naming, transport protocol, schema format, wire format, serialization format, and response format may vary across embodiments and deployments.

The disclosed invention is not limited to canonical nucleic-acid inputs. In some embodiments, the sequence contains canonical DNA or RNA residues. In some embodiments, the sequence contains modified residues, analogs, ambiguous residues, tagged residues, lesion-bearing residues, therapeutic substitutions, or another non-canonical content. In some embodiments, the disclosed framework is applied at the residue level. In some embodiments, the disclosed framework is applied at the codon level, motif level, grouped-window level, or another derived-unit level. In some embodiments, the disclosed framework is applied to RNA polymerase. In some embodiments, the disclosed framework is applied to DNA polymerase, reverse transcriptase, ribosome-associated translation, or another processive molecular machine.

The disclosed thresholds, scores, and decision rules are also illustrative and non-limiting. A pause site may be called using a fixed threshold, a relative threshold, a percentile threshold, an adaptive threshold, a local-maximum rule, a cluster rule, a ranked top-N rule, a feature-aware rule, or another deterministic policy. A pause-related output may be represented as a rate, dwell time, normalized pause density, local score, cluster score, ratio, difference, cumulative burden, regional burden, annotation-linked metric, or another derived measure. The invention is therefore not limited to a single output metric or a single decision criterion, even where one metric may be preferred in a particular embodiment.

The disclosed profile-related quantities are also not limited to a single representation. In some embodiments, nu0 is a scalar value. In some embodiments, nu0 is profile-dependent, condition-dependent, state-dependent, or time-dependent. In some embodiments, a correction term is zero. In some embodiments, a correction term is non-zero. In some embodiments, a correction term is an integer, a half-integer, a rational value, or another quantized value. In some embodiments, one or more correction terms are stored in a lookup table. In some embodiments, one or more correction terms are generated by a deterministic rule or selected from a policy bundle. In some embodiments, the same sequence may be evaluated under multiple profile bundles, temperatures, correction policies, annotation policies, or output policies.

The disclosed figures are schematic and are not necessarily drawn to scale. A depicted block, pathway, relationship, or connection is intended to illustrate one or more functional relationships and should not be interpreted as requiring a particular physical arrangement, geometric proportion, timing relationship, line style, data bus structure, memory boundary, network boundary, or deployment boundary unless expressly stated otherwise. A reference numeral used in connection with one embodiment or one figure may identify a representative structure or function and does not necessarily require that every embodiment contain a physically separate structure corresponding to that numeral.

Where a value, constant, equation, mapping, or output is described as preferred, representative, illustrative, exemplary, or in some embodiments, such description should not be construed as disclaiming alternatives unless such exclusion is expressly stated. Similarly, where the specification describes a feature as being used in “preferred embodiments,” that description is intended to identify one presently preferred implementation and not to limit the invention to that implementation. Ranges, example values, benchmark values, and implementation examples are provided to illustrate how the invention may be practiced and are not intended to restrict the claims to those exact values unless expressly recited in the claims.

The disclosed formal verification embodiment is likewise optional and non-limiting. The invention may be practiced with or without theorem proving, proof-carrying artifacts, formal certification, symbolic verification, or deployment attestations. Where formal verification is used, it may be applied to all, some, or only selected parts of the disclosed system. Where formal verification is not used, the substantive computational framework remains within the scope of the invention.

Accordingly, all suitable modifications, substitutions, reconfigurations, equivalent structures, equivalent computations, equivalent data representations, equivalent deployment models, and equivalent process flows that fall within the scope of the appended claims are intended to be embraced by the invention.

1. A computer-implemented method for determining sequence-dependent pause behavior of a processive molecular machine, the method comprising: receiving, by one or more processors, a nucleic-acid sequence comprising a plurality of ordered positions; receiving, by the one or more processors, a temperature and a selected profile associated with the processive molecular machine; assigning, by the one or more processors, a core ladder index to each of the ordered positions according to residue identity; determining, by the one or more processors, an effective ladder index for each of the ordered positions based at least in part on the core ladder index; computing, by the one or more processors, a position-resolved forward rate for each of the ordered positions as a function of the effective ladder index, the temperature, and a coherence-scale barrier quantity; computing, by the one or more processors, a position-resolved dwell time for each of the ordered positions from the forward rate; computing, by the one or more processors, a pause metric for the ordered positions from the dwell times; and outputting, by the one or more processors, data representing the pause metric for at least a portion of the nucleic-acid sequence.

2. The method of claim 1, wherein assigning the core ladder index comprises assigning:
n(A)=0,
n(T)=1,
n(U)=1,
n(G)=2, and
n(C)=3.

3. The method of claim 1, wherein determining the effective ladder index comprises determining: n_eff(i)=n(i)+delta_nn(i)+delta_mod(i)+delta_ctx(i)+delta_ext(i), where n(i) is the core ladder index for position i, and wherein delta_nn(i), delta_mod(i), delta_ctx(i), and delta_ext(i) each comprise a deterministic correction term and are independently selectable to be zero such that n_eff(i)=n(i) when no correction is applied.

4. The method of claim 3, wherein at least one of delta_nn(i), delta_mod(i), delta_ctx(i), or delta_ext(i) comprises a nearest-neighbor correction, a modified-residue correction, a higher-order context correction, or an annotation-based correction.

5. The method of claim 1, wherein computing the position-resolved forward rate comprises computing: k(i; p, T)=nu0(p)*exp(−(n_eff(i)*E_coh)/(k_B*T)), where i identifies an ordered position in the nucleic-acid sequence, p identifies the selected profile, n_eff(i) is the effective ladder index for position i, nu0(p) is an attempt-frequency term associated with profile p, E_coh is the coherence-scale barrier quantity, k_B is Boltzmann's constant, and T is an absolute temperature.

6. The method of claim 5, wherein computing the position-resolved dwell time comprises computing: tau(i; p, T)=1/k(i; p, T).

7. The method of claim 6, wherein computing the pause metric comprises computing a normalized pause density according to: sigma(i; p, T)=tau(i; p, T)/sum_{j=1 to L} tau(j; p, T), where L is a sequence length.

8. The method of claim 7, wherein the selected profile is associated with an attempt frequency nu0(p) that is constant across the ordered positions, and wherein the normalized pause density sigma(i; p, T) is invariant to a constant profile-wide scaling of nu0(p).

9. The method of claim 1, wherein the coherence-scale barrier quantity comprises E_coh derived from:
J(xy)+J(x/y)=2J(x)J(y)+2J(x)+2J(y),
J(x)=0.5(x+1/x)−1,
phi=(1+sqrt(5))/2,X_opt=phi/pi, andE_coh=E_Planck*X_opt\hat{ }101,where pi is the mathematical constant equal to the ratio of a circle's circumference to its diameter.

10. The method of claim 1, wherein the processive molecular machine comprises an RNA polymerase, a DNA polymerase, a reverse transcriptase, or a ribosome-associated translation system.

11. The method of claim 1, further comprising parsing the nucleic-acid sequence from a sequence record comprising plain-text sequence content, FASTA content, FASTQ content, GenBank content, streamed base calls, or a coordinate-derived subsequence, and canonicalizing the nucleic-acid sequence prior to assigning the core ladder index.

12. The method of claim 1, wherein the pause metric comprises at least one of a forward-rate value, a dwell-time value, a normalized pause-density value, a local window score, a cluster score, a differential score, a ratio score, a cumulative burden value, or a regional burden value.

13. The method of claim 1, further comprising identifying a pause site or pause region by applying thresholding, ranking, clustering, local-maximum detection, or windowed scoring to the pause metric.

14. The method of claim 1, further comprising comparing a baseline condition and a perturbed condition to generate a comparative pause output, wherein the perturbed condition comprises at least one of a modified residue, a nucleotide analog, a chemical perturbation, a different temperature, a different selected profile, or a different correction policy.

15. The method of claim 1, wherein receiving the nucleic-acid sequence comprises receiving a stream of sequence symbols or base calls incrementally, and wherein outputting the pause metric comprises outputting a rolling or partial pause representation prior to receipt of an entire sequence.

16. The method of claim 1, further comprising generating a precomputed pause atlas for a plurality of sequences or for at least a portion of a genome, transcriptome, or sequence library.

17. A system for determining sequence-dependent pause behavior of a processive molecular machine, the system comprising: one or more processors; and a non-transitory memory storing instructions that, when executed by the one or more processors, cause the one or more processors to: receive a nucleic-acid sequence comprising a plurality of ordered positions; receive a temperature and a selected profile associated with the processive molecular machine; assign a core ladder index to each of the ordered positions according to residue identity; determine an effective ladder index for each of the ordered positions based at least in part on the core ladder index; compute a position-resolved forward rate for each of the ordered positions as a function of the effective ladder index, the temperature, and a coherence-scale barrier quantity; compute a position-resolved dwell time for each of the ordered positions from the forward rate; compute a pause metric for the ordered positions from the dwell times; and output data representing the pause metric for at least a portion of the nucleic-acid sequence.

18. The system of claim 17, wherein the instructions further cause the one or more processors to expose the output data through an application programming interface, store the output data in a result store, or both.

19. A non-transitory computer-readable medium storing instructions that, when executed by one or more processors, cause the one or more processors to: receive a nucleic-acid sequence comprising a plurality of ordered positions; receive a temperature and a selected profile associated with a processive molecular machine; assign a core ladder index to each of the ordered positions according to residue identity; determine an effective ladder index for each of the ordered positions based at least in part on the core ladder index; compute a position-resolved forward rate for each of the ordered positions as a function of the effective ladder index, the temperature, and a coherence-scale barrier quantity; compute a position-resolved dwell time for each of the ordered positions from the forward rate; compute a pause metric for the ordered positions from the dwell times; and output data representing the pause metric for at least a portion of the nucleic-acid sequence.

20. The non-transitory computer-readable medium of claim 19, wherein the instructions further cause the one or more processors to identify a pause site or pause region, compare a baseline condition and a perturbed condition, generate a precomputed pause atlas, or any combination thereof.
